(19) United States
(12) Patent Application Publication
(10) Pub. No.: US 2025/0144336 A1
(43) Pub. Date: May 8, 2025

(54) PATIENT GAS DELIVERY SYSTEMS AND METHODS

(71) Applicant: Fisher & Paykel Healthcare Limited, Auckland (NZ)

(72) Inventors: Adam John DARBY, Auckland (NZ); Mark Samuel HAMILTON, Auckland (NZ); David John SIMS, Auckland (NZ); Sean Joel BABBAGE, Auckland (NZ); Simei Gomes WYSOSKI, Auckland (NZ); Alan John GRIMMER, Auckland (NZ); Johannes Nicolaas BOTHMA, Auckland (NZ); Zak Jake FLINTOFF, Auckland (NZ); Frederick David Allan WRIGHT, Auckland (NZ); Madison Brooke CHU, Auckland (NZ)

(21) Appl. No.: 18/837,541

(22) PCT Filed: Feb. 13, 2023
(86) PCT No.: PCT/IB2023/051252

§ 371 Date: Aug. 9, 2024

Related U.S. Application Data
(60) Provisional application No. 63/309,145, filed on Feb. 11, 2022.

(51) Int. Cl. A61M 16/00

(52) U.S. Cl. CPC A61M 16/024; A61M 16/0003; A61M 2016/0027; A61M 2016/0033; A61M 2202/0007; A61M 2202/02; A61M 2205/103; A61M 2205/3334; A61M 2205/3344; A61M 2205/3365; A61M 2205/50; A61M 2240/00

(57) ABSTRACT

A device arranged to be in fluid communication with a patient interface arranged to be in communication with an airway of a patient during respiratory therapy, the device comprising: an inlet for receiving a breathable gas from a respiratory apparatus; an outlet for conveying the breathable gas to the patient interface; a body portion extending between the inlet and the outlet, wherein the body portion includes a trigger port, and one or more sensors, wherein output of the one or more sensors is used to determine a status of the trigger port.

### FIELD

The present disclosure generally relates to patient gas delivery systems and methods.

### BACKGROUND

Positive End Expiratory Pressure (PEEP) and/or Peak Inspiratory Pressure (PIP) can be controllably provided to a patient during respiration, resuscitation or assisted respiration (ventilation).

PEEP is a pressure delivered to the patient throughout the expiratory phase of positive pressure ventilation, resuscitation, or assisted respiration. PIP is a desired highest pressure provided to the patient during the inspiratory phase of positive pressure ventilation, resuscitation, or assisted respiration. The patients may be neonates or infants who require breathing assistance or resuscitation. In applying PEEP or PIP, the patient's upper airway and lungs are held open by the applied pressure.

Some respiratory therapy systems, such as those which may be used in infant resuscitation, rely on a T-piece device to manually adjust the pressure of breathable gas delivered to a patient. Those existing infant resuscitation respiratory systems which incorporate a T-piece device are often pneumatic and work from a fixed gas flow source, typically a wall-source, with a pre-set or a manually adjustable flow rate. In use, the gas flow source supplies a flow of breathable gas to a respiratory apparatus (for example, a resuscitation device), and the respiratory apparatus then supplies the breathable gas to the patient via interconnected gas supply tubes, a T-piece device, and a suitable patient interface. Typically there is a flow meter used to control or monitor the flow rate of the gas flow from the fixed gas flow source. In addition, the respiratory apparatus includes user controls which allow an operator to set and monitor the pressure of the breathable gas delivered to the patient. Some of the existing respiratory apparatuses are constant flow rate devices, that is, the respiratory apparatus does not adjust the flow rate of the breathable gas it delivers to the patient. Instead, the fixed gas flow source is operated at a constant flow rate, and the respiratory apparatus includes a pressure relief valve and a calibrated flow resistance which can be adjusted to achieve the desired PEEP and PIP. In these systems, an orifice is usually provided in the T-piece device which can be occluded or unoccluded by an operator of the respiratory system. When the orifice is occluded, the delivered pressure is determined by the pressure regulating valve in the respiratory apparatus. This is the “PIP” pressure setting. When the orifice is open, the delivered pressure is determined by a pressure regulating valve inside the T-piece device. This is the “PEEP” pressure setting. As the orifice is repeatedly occluded and unoccluded, PIP and PEEP are delivered to the patient to deliver breaths to the patient.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

### SUMMARY OF THE INVENTION

In a first aspect, the present disclosure provides a device arranged to be in fluid communication with a patient interface arranged to be in communication with an airway of a patient during respiratory therapy, the device comprising:
an inlet for receiving a breathable gas from a respiratory apparatus;
an outlet for conveying the breathable gas to the patient interface;
a body portion extending between the inlet and the outlet, wherein the body portion includes a trigger port, and
one or more sensors, wherein output of the one or more sensors is used to determine a status of the trigger port.

In some configurations, the body portion includes one or more sensing ports.

In a second aspect, the present disclosure provides a device arranged to be in fluid communication with a patient interface arranged to be in communication with an airway of a patient during respiratory therapy, the device comprising:
an inlet for receiving a breathable gas from a respiratory apparatus;
an outlet for conveying the breathable gas to the patient interface, and
a body portion extending between the inlet and the outlet, wherein the body portion includes a trigger port, and one or more sensing ports, arranged to be fluidly connected to one or more sensors, wherein output of the one or more sensors is used to determine a status of the trigger port.

In some configurations, the body portion includes a first member, wherein the trigger port is formed in a wall or an end of the first member.

In some configurations, the body portion includes a second member, which is of a substantially hollow configuration creating a passage for the breathable gas to flow through.

In some configurations, the second member is configured to allow attachment to an inlet of a patient interface.

In some configurations, the first and the second members are joined together permanently.

In some configurations, the first and second members are joined together using ultrasonic welding, adhesive, or overmolding.

In some configurations, the first and second members are removably joined together.

In some configurations, the one or more sensors include one or more pressure and/or flow rate sensors.

In some configurations, the one or more sensors include at least one differential pressure sensor.

In some configurations, the at least one differential pressure sensor includes a flow restriction in a gas flow path, wherein the flow restriction is arranged to create a differential pressure on either side of the flow restriction.

In some configurations, the flow restriction may include one or more deflectable flaps, a variable orifice membrane/diaphragm, and similar there of.

In some configurations, a deflectable flap is positioned between the first and second members.

In some configurations, the one or more sensors at least partially extend into an internal cavity of the body portion.

In some configurations, the one or more sensors at least partially extend into the one or more sensing ports.

In some configurations, the one or more sensors are accommodated within the body portion of the device.

In some configurations, at least one of the one or more sensors is located outside the body portion, and is arranged to receive a flow of the breathable gas from the one or more sensing ports.

In some configurations, the one or more sensing ports include two pressure sensing ports, which extend outwardly from the wall of the body portion.

In some configurations, at least one of the one or more sensors is used to measure a flow rate of breathable gas delivered to the patient.

In some configurations, at least one of the one or more sensors is used to measure a pressure of the breathable gas in the device, or in the patient interface.

In some configurations, the status of the trigger port includes an open state, and a closed state.

In some configurations, the trigger port is in a closed state if it is blocked by a finger of an operator.

In some configurations, the trigger port is in an open state if it is not blocked by the finger of an operator, such that an airflow within the device is allowed to exit to ambient air.

In some configurations, the trigger port is an orifice formed in the wall the body portion.

In some configurations, the trigger port is a circular orifice formed in the wall of the body portion.

In some configurations, the output of the one or more sensors is received or obtained by a controller, which determines the status of the trigger port, and/or a change in the status of the trigger port based on the output.

In some configurations, the controller controls operation of the respiratory apparatus such that the breathable gas is delivered to the patient at a suitable pressure.

In some configurations, the controller controls operation of the respiratory apparatus by setting a motor speed of a flow generator.

In some configurations, the controller sends a control signal to the respiratory apparatus when there is a change in the status of trigger port.

In some configurations, the device includes a coupling mechanism allowing it to be removably connected to the patient interface.

In some configurations, the device receives the breathable gas from the respiratory apparatus via a conduit.

In some configurations, the conduit is inserted into the inlet of the device.

In some configurations, the device is a T-piece device.

In a further aspect, the present disclosure provides a device circuit, comprising:
a device according to the first or second aspect of the present disclosure;
a conduit which is coupled to the inlet of the device at a first end, and arranged to connect to a gas outlet of a respiratory apparatus at a second end to receive a flow of breathable gas therefrom.

In some configurations, the conduit includes a connector at the second end for connecting to the respiratory apparatus.

In some configurations, the connector comprises a tapered connection portion arranged to be inserted into an outlet of the respiratory apparatus.

In some configurations, the conduit has a length of 1 to 2 meters, or approximately 1.5 to 2 meters, or 1.6 meter.

In some configurations, the conduit is non-removably coupled to the inlet of the device.

In a third aspect, the present disclosure provides an interface assembly for use in respiratory therapy, the interface assembly includes:
a device according to any one or more of the first or second aspect of this disclosure, and
a patient interface arranged to be in communication with an airway of a patient during respiratory therapy.

In a fourth aspect, the present disclosure provides a gas delivery system for delivering a breathable gas to a patient, comprising:
a respiratory apparatus including a flow generator, a controller arranged to control the flow generator, and one or more sensors configured to determine a source flow rate from the flow generator (Fs), and an interface gas flow rate (Fm);
a conduit assembly for delivering the breathable gas to the patient from the flow generator;
an interface assembly arranged to receive the breathable gas from the conduit assembly, and convey the breathable gas to the patient, wherein the interface assembly comprises a trigger port;
   wherein the controller is arranged to: calculate a difference between the source flow rate and the interface gas flow rate, determine a status of the trigger port based on the difference, and operate the flow generator to provide the breathable gas to the patient at a pressure based on the determined status of the trigger port.

In some configurations, the interface assembly includes:
a patient interface in communication with an airway of a patient, and
a device fluidly coupled to the patient interface and the conduit assembly, for conveying the breathable gas from the conduit assembly to the patient interface.

In some configurations, the one or more sensors include one or more of a flow rate sensor, pressure sensor, differential pressure sensor, mass flow sensor, ultrasonic flow sensor, and/or thermistors.

In some configurations, the differential pressure sensor includes a flow restriction in a gas flow path, wherein the flow restriction is arranged to create a differential pressure on either side of the flow restriction.

In some configurations, the flow restriction may include one or more deflectable flaps, a variable orifice membrane/diaphragm, and similar there of.

In some configurations, the differential pressure sensor includes two pressure sensing elements arranged to measure a first and a second pressure of the breathable gas in the device.

In some configurations, the pressure sensing elements comprise pressure sensing tubes.

In some configurations, the device includes two pressure sensing ports, wherein the pressure sensing elements are arranged to take pressure measurements via the pressure sensing ports.

In some configurations, the pressure sensing elements are arranged measure a pressure differential on either side of the deflectable flap.

In some configurations, the trigger port is located in a wall of the device.

In some configurations, the device is a T-piece device.

In some configurations, the status of the trigger port includes at least a closed state and an open state.

In some configurations, if the trigger port is blocked by an object, to restrict or prevent gas flow via the trigger port, the status of the trigger port is closed, whereas if the trigger port is not blocked, the status of the trigger port is open.

In some configurations, the controller is arranged to:
compare the difference with a first and/or a second threshold flow rates (Fa, Fb) and
determine the status of trigger port based on the comparison, wherein the trigger port is either in an open or a closed state.

In some configurations, the controller is arranged to:
obtain a previous status of the trigger port from a memory;
compare the difference with a first threshold flow rate Fa, if the previous status of the trigger port is in an open state, or comparing the difference with a second threshold flow rate Fb, if the previous status of the trigger port is in a closed state;
determine a current status of the trigger port based on the comparison, and
determine if the status of the trigger port has changed by comparing the previous status with the current status.

In some configurations, the trigger port is in a closed state if the difference between the source flow rate and the interface gas flow rate is smaller than the first threshold flow rate (ΔF<Fa).

In some configurations, the trigger port is in an open state if the difference is greater than the second threshold flow rate (ΔF>Fb).

In some configurations, the second threshold flow rate is calculated based on interface pressure.

In some configurations, the second threshold flow rate is calculated from K*sqrt(Pm), wherein Pm is interface pressure, and K is a coefficient determined experimentally or during a calibration stage.

In some configurations, the second threshold flow rate is within a range of 0.5 to 10 Litres per minute (L/min).

In some configurations, the second threshold flow rate is set at a constant level.

In some configurations, the second threshold flow rate is 1 L/min.

In some configurations, the first threshold flow rate is determined based on interface pressure.

In some configurations, the first threshold flow rate is calculated from Fa=J*sqrt(Pm), wherein Pm is interface pressure, and J is a coefficient determined experimentally or during a calibration stage.

In some configurations, the first threshold flow rate is within a range of 0.5 to 10 L/min.

In some configurations, the first threshold flow rate is set at a constant level.

In some configurations, the first threshold flow rate is set at 1 L/min.

In some configurations, the controller is arranged to operate the flow generator to deliver a breathable gas to the patient at a first pressure when the trigger port is determined to be in a closed state, and at a second pressure when the trigger port is determined to be in an open state.

In some configurations, the first pressure is higher than the second pressure.

In some configurations, the first pressure is a high pressure, and a second pressure is a low pressure.

In some configurations, the high pressure gas flow corresponds to peak inspiratory pressure (PIP) and the low pressure gas flow corresponds to positive end expiratory pressure (PEEP).

In some configurations, for an infant or neonatal patient, PEEP is equivalent to 1, 2, 3, 4, 5, 6, 7, or 8 cm H₂O, and PIP is equivalent to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O.

In some configurations, the controller operates the flow generator by setting a motor speed of the flow generator.

In some configurations, the motor speed of the flow generator is adjusted over a predefined time interval.

In some configurations, the predetermined time interval is between 100 to 400 ms, or between 100 to 300 ms, or between 100 to 200 ms, or approximately 150 ms.

In some configurations, the flow generator is fluidly coupled to a source of breathable gas supply, which may be a wall mounted gas source.

In some configurations, the system includes a humidifier for adding humidity to the breathable gas before it is conveyed to the patient.

In some configurations, the system is used for delivering the breathable gas to resuscitate the patient, or to deliver a positive pressure ventilation (PPV) therapy to the patient.

In a fifth aspect, the present disclosure provides a method of controlling a gas delivery system, by:
determining a source gas flow;
determining a delivery gas flow;
calculating a difference between the source gas flow and the delivery gas flow, and
causing the gas delivery system to deliver a breathable gas to the patient at a pressure based on the calculated difference between the source and delivery gas flow.

In some configurations, the step of determining a source gas flow comprises:
receiving or obtaining measurements from one or more first sensors, and
determining a first flow rate based on the measurements, being the flow rate of source gas flow (Fs).

In some configurations, the step of determining a delivery gas flow comprises:
receiving or obtaining measurements from one or more second sensors, and
determining a second flow rate based on the measurements, being the flow rate of the breathable gas supplied to a patient interface (Fm).

In some configurations, the step of calculating a difference between the source gas flow and delivery gas flow includes:
calculating a difference between the first and second flow rates (ΔF=Fs−Fm).

In some configurations, the method further includes:
comparing the difference between the first and second flow rates with a first and/or a second threshold flow rate (Fa, Fb), and
determining a status of a trigger port based on the comparison.

In some configurations, the status of the trigger port includes: an open state, or a closed state.

In some configurations, the method includes:
obtaining a previous status of a trigger port, wherein the previous status of the trigger port includes an open state or a closed state;
comparing the difference between the first and second flow rates with a first threshold flow rate Fa, if previous status of the trigger port is in an open state, or comparing the difference between the first and second flow rates with a second threshold flow rate Fb, if the previous status of the trigger port is in a closed state;
determining a current status of the trigger port based on the comparison, and
determining if status of the trigger port has changed by comparing the previous status with the current status.

In some configurations, the trigger port is in the closed state if the difference is smaller than the first threshold flow rate (ΔF<Fa).

In some configurations, the trigger port is in the open state if the difference is greater than the second threshold flow rate (ΔF>Fb).

In some configurations, the second threshold flow rate is calculated based on interface pressure.

In some configurations, the second threshold flow rate is calculated from K*sqrt (Pm), wherein Pm is the interface pressure, and K is a coefficient determined experimentally or during a calibration stage.

In some configurations, the second threshold flow rate is within a range of 0.5 to 10 L/min.

In some configurations, the second threshold flow rate is set at a constant level.

In some configurations, the second threshold flow rate is set at a constant level of 1 L/min.

In some configurations, the first threshold flow rate is determined based on interface pressure.

In some configurations, the first threshold flow rate is calculated from Fa=J*sqrt(Pm), wherein Pm is interface pressure, and J is a coefficient determined experimentally or during a calibration stage.

In some configurations, the first threshold flow rate is within a range of 0.5 to 10 L/min.

In some configurations, the first threshold flow rate is set at a constant level.

In some configurations, the first threshold flow rate is set at a constant level of 1 L/min.

In some configurations, the method further includes:
operating the gas delivery system to deliver the breathable gas to the patient at a first pressure, if the trigger port is determined to be in a closed state, and
operating the gas delivery system to deliver the breathable gas to the patient at a second pressure, if the trigger port is determined to be in an open state.

In some configurations, the method further includes:
operating the gas delivery system to continue to deliver the breathable gas to the patient at the first or a second pressure to the patient, until the status of the trigger port changes.

In some configurations, the first pressure is higher than the second pressure.

In some configurations, the first pressure corresponds to peak inspiratory pressure (PIP) and the second pressure corresponds to positive end expiratory pressure (PEEP).

In some configurations, for an infant or neonatal patient, PEEP is equivalent to 1, 2, 3, 4, 5, 6, 7, or 8 cm H₂O, and PIP is equivalent to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O.

In some configurations, the step of operating the gas delivery system to deliver the breathable gas to the patient at the first or second pressure includes:
sending a control signal to a flow generator of the system.

In some configurations, the flow generator is operated by controlling a motor speed of the flow generator.

In some configurations, the flow generator is operated by controlling a speed of a blower fan.

In some configurations, the motor speed is changed over a predetermined time interval until the delivered breathable gas reaches the first or second pressure.

In some configurations, the predetermined time interval is between 100 to 400 ms, or between 100 to 300 ms, or between 100 to 200 ms, or approximately 150 ms.

In some configurations, the method further includes:
causing the trigger port to be in a closed state by substantially reducing or preventing air flow via the trigger port, and
causing the trigger port to be in an open state by allowing air flow via the trigger port.

In some configurations, the step of causing the trigger port to be in a closed state comprises blocking the trigger port with an object, and the step of causing the trigger port to be in an open state comprises removing the object from the trigger port.

In some configurations, the object is a finger of an operator.

In some configurations, the step of determining a second flow rate includes:
measuring a first pressure of the breathable gas at a first location;
measuring a second pressure of the breathable gas at a second location;
calculating a pressure differential between the first and the second pressure;
calculating the flow rate of the breathable gas supplied to the patient interface (Fm) based on the pressure differential.

In some configurations, the first location is at or near an inlet of a device, where it receives the breathable gas from a conduit assembly in fluid communication with the flow generator.

In some configurations, the second location is at or near an outlet of the device, where it conveys the breathable gas to the patient interface.

In some configurations, the second pressure is the interface pressure.

In a seventh aspect, the present disclosure provides a method of delivering a breathable gas to a patient via an interface assembly, said interface assembly including a trigger port, comprising:
determining a status of the trigger port, by determining a source gas flow generated by a gas delivery system and a delivery gas flow delivered to the interface assembly,
causing the gas delivery system to deliver a breathable gas to the patient at a pressure based on the determined status of the trigger port.

In some configurations, the step of determining a source gas flow comprises:
receiving or obtaining measurements from one or more first sensors, and
determining a first flow rate based on the measurements, being the flow rate of source gas flow (Fs).

In some configurations, the step of determining a delivery gas flow comprises:
receiving or obtaining measurements from one or more second sensors, and
determining a second flow rate based on the measurements, being the flow rate of the breathable gas supplied to a patient interface (Fm).

In some configurations, the method further incudes: calculating a difference between the first and second flow rates (ΔF=Fs−Fm).

In some configurations, the method further includes:
comparing the difference between the first and second flow rates with a first and/or a second threshold flow rate (Fa, Fb) and
determining a status of a trigger port based on the comparison.

In some configurations, the status of the trigger port includes: an open state, or a closed state.

In some configurations, the method includes:
obtaining a previous status of a trigger port, wherein the previous status of the trigger port includes an open state or a closed state;
comparing the difference between the first and second flow rates with a first threshold flow rate Fa, if the previous status of the trigger port is in an open state, or comparing the difference between the first and second flow rates with a second threshold flow rate Fb, if the previous status of the trigger port is in a closed state;
determining a current status of the trigger port based on the comparison, and
determining if status of the trigger port has changed by comparing the previous status with the current status.

In some configurations, the trigger port is in the closed state if the difference is smaller than the first threshold flow rate (ΔF<Fa).

In some configurations, the trigger port is in the open state if the difference is greater than the second threshold flow rate (ΔF>Fb).

In some configurations, the second threshold flow rate is calculated based on interface pressure.

In some configurations, the second threshold flow rate is calculated from K*sqrt(Pm), wherein Pm is interface pressure, and K is a coefficient determined experimentally or during a calibration stage.

In some configurations, the second threshold flow rate is within a range of 0.5 to 10 L/min.

In some configurations, the second threshold flow rate is set at a constant level.

In some configurations, the second threshold flow rate is set at a constant level of 1 L/min.

In some configurations, the first threshold flow rate is determined based on interface pressure.

In some configurations, the first threshold flow rate is calculated from Fa=J*sqrt(Pm), wherein Pm is interface pressure, and J is a coefficient determined experimentally or during a calibration stage.

In some configurations, the first threshold flow rate is within a range of 0.5 to 10 L/min.

In some configurations, the first threshold flow rate is set at a constant level.

In some configurations, the first threshold flow rate is set at a constant level of 1 L/min.

In some configurations, the method further includes:
operating the gas delivery system to deliver a breathable gas to the patient at a first pressure, if the trigger port is determined to be in a closed state, and
operating the gas delivery system to deliver a breathable gas to the patient at a second pressure, if the trigger port is determined to be in an open state.

In some configurations, the method further includes:
operating the gas delivery system to continue to deliver the breathable gas to the patient at the first or a second pressure to the patient, until the status of the trigger port changes.

In some configurations, the first pressure is higher than the second pressure.

In some configurations, the first pressure corresponds to peak inspiratory pressure (PIP) and the second pressure corresponds to positive end expiratory pressure (PEEP).

In some configurations, for an infant or neonatal patient, PEEP is equivalent to 1, 2, 3, 4, 5, 6, 7, or 8 cm H₂O, and PIP is equivalent to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O.

In some configurations, the step of causing the gas delivery system to deliver the breathable gas to the patient at the first or second pressure includes:
sending a control signal to a flow generator of the system.

In some configurations, the flow generator is operated by controlling a motor speed of the flow generator.

In some configurations, the flow generator is operated by controlling a speed of a blower fan.

In some configurations, the motor speed is changed over a predetermined time interval until the delivered breathable gas reaches the first or second pressure flow.

In some configurations, the predetermined time interval is between 100 to 400 ms, or between 100 to 300 ms, or between 100 to 200 ms, or approximately 150 ms.

In some configurations, the method further includes:
causing the trigger port to be in a closed state by substantially reducing or preventing air flow via the trigger port, and
causing the trigger port to be in an open state by allowing air flow via the trigger port.

In some configurations, the step of causing the trigger port to be in a closed state comprises blocking the trigger port with an object, and the step of causing the trigger port to be in an open state comprises removing the object from the trigger port.

In some configurations, the object is a finger of an operator.

In some configurations, the step of determining a second flow rate includes:
measuring a first pressure of the breathable gas at a first location;
measuring a second pressure of the breathable gas at a second location;
calculating a pressure differential between the first and the second pressure;
calculating the flow rate of the breathable gas supplied to the patient interface (Fm) based on the pressure differential.

In some configurations, the first location is at or near an inlet of a device, where it receives the breathable gas from a conduit assembly in fluid communication with the flow generator.

In some configurations, the second location is at or near an outlet of the device, where it conveys the breathable gas to the patient interface.

In some configurations, the second pressure is the interface pressure Pm.

In an eighth aspect, the present disclosure provides a gas delivery system for delivering a breathable gas to a patient, comprising:
a respiratory apparatus including a flow generator, a controller arranged to control the flow generator, and at least one sensor proximate the flow generator, the at least one sensor configured to determine at least a source flow rate from the flow generator (Fs),
a conduit assembly for delivering the breathable gas to the patient from the flow generator;
an interface assembly arranged to receive the breathable gas from the conduit assembly, and convey the breathable gas to the patient, wherein the interface assembly comprises a trigger port;
wherein the controller is arranged to determine a status of the trigger port by comparing the determined source flow rate (Fs) to a threshold flow rate (Fth), and operate the flow generator to provide the breathable gas to the patient at a pressure based on the determined status of the trigger port.

In some configurations, the status of the trigger port is either open or closed.

In some configurations, pressure is provided at either a PIP state or a PEEP state; the PIP state being higher than the PEEP state; the PIP state corresponding to the trigger port being closed, and the PEEP state corresponding to the trigger port being open.

In some configurations, the controller is further configured to determine a pressure (P) at a point in the system.

In some configurations, the threshold flow rate (Fth) is related to L*sqrt(P), where L is a coefficient, and P is a mathematically determined pressure at a patient interface of the interface assembly.

In some configurations, L is related to a conductance value associated with two or more flow egress points/scenarios of the system.

In some configurations, L is related to a conductance value associated with the trigger port and a conductance value associated with a minimum expected flow through the system with the trigger port closed and no flow egress through the patient interface.

In some configurations, L is related to a conductance value associated with a minimum expected flow through the system with the trigger port closed and no flow egress through the patient interface, and with a conductance value associated with maximum flow egress through the patient interface during delivery of PIP.

In some configurations, the threshold flow rate (Fth) is further related to a rate at which flow is entering/exiting the patient's lungs.

In some configurations, if Fs<Fth, then the trigger port is determined to be in the closed state.

In some configurations, if Fs>Fth, then the trigger port is determined to be in the open state.

In some configurations, the determination of the status of the trigger port further comprises a hysteresis analysis, wherein a prior status of the trigger port is considered in determining the status of the trigger port.

In some configurations, the system is further configured to detect one or more of:
disconnection, and/or improper/incomplete connection (collectively a “connection problem”), of the patient interface;
disconnection of one or more other components of the system, such as the conduit or a humidifier; and/or
blockage in or of the system.

In some configurations, the system is configured to detect a connection problem of the patient interface, wherein the system is configured to determine an excessive flow threshold (Fₗₑₐₖₜₕ) indicative of a patient interface connection problem.

In some configurations, Fₗₑₐₖₜₕ is dependent on: one or more conductance values; and pressure.

In some configurations, Fₗₑₐₖₜₕ is further dependent on a rate at which flow is entering/exiting the patient's lungs.

In some configurations, when dependent on claim 71, wherein the system determines a patient interface connection problem to be present when the source flow rate (Fs) is greater than Fₗₑₐₖₜₕ.

In some configurations, when dependent on claim 36, wherein the system determines a patient interface connection problem to be present when the interface gas flow rate (Fm) is greater than Fₗₑₐₖₜₕ.

In some configurations, wherein, if a patient interface connection problem is detected, the system is configured to do one or more of:
set the source flow rate (Fs) to a fixed flow rate;
set a motor speed of the flow generator to a fixed speed;
set the pressure at the flow generator to a fixed pressure; and/or
generate an alarm or alert.

In some configurations, the system is configured to detect disconnection of one or more other components of the system, such as the conduit or the humidifier; the system being configured to: determine a conductance corresponding to the one or more other components of the system; determine an overall conductance of the system; and detect disconnection of said one or more other components of the system if the overall conductance of the system is greater than the conductance corresponding to the one or more other components.

In some configurations, if disconnection of said one or more other components is detected, the system is configured to: set a fixed motor speed; and/or generate an alarm or alert.

In some configurations, the system is configured to detect blockage in or of the system, wherein the system is configured to determine a threshold flow value for use in detecting blockage.

In some configurations, the threshold flow value corresponds to a minimum expected flow through the system with the trigger port closed and no flow egress through the patient interface; and wherein, if the source flow (Fs) is lesser than the threshold flow value, blockage is detected.

In some configurations, the threshold flow value is derived from a combination of: a conductance associated with a minimum expected flow through the system with the trigger port closed and no flow egress through the patient interface; and the pressure at the patient interface; and wherein, if the source flow (Fs) is lesser than the threshold flow value, blockage is detected.

In some configurations, the threshold flow value is further derived from an actual or approximated lung compliance of the patient.

In some configurations, if blockage in or of the system is detected, the system is configured to: set a fixed motor speed; and/or generate an alarm or alert.

Further aspects and advantages of certain embodiments of the disclosure will become apparent from the ensuing description, which is given by way of example only.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 shows an example of a gas delivery system according to one embodiment of the present disclosure;

FIG. 2 shows an example of a conduit assembly and an interface assembly according to one embodiment of the present disclosure;

FIGS. 3A and 3B each shows an example of a device according to one embodiment of the present disclosure;

FIGS. 4A and 4B each shows an alternative example of a device according to one embodiment of the present disclosure;

FIG. 5 shows cross sectional diagrams of an embodiment of a valve used to occlude or unocclude a trigger port of a device, with its actuator in a range of different positions;

FIG. 6 shows side on exterior views of the valve of FIG. 5;

FIG. 7 shows a simplified schematic diagram of a gas delivery system according to one embodiment;

FIG. 8 shows another simplified schematic diagram of a gas delivery system according to one embodiment;

FIG. 9 shows a schematic of a control logic implemented in an embodiment of a controller;

FIG. 10 shows a schematic of another control logic implemented in an embodiment of a controller;

FIG. 11 shows yet another example of a control logic implemented in an embodiment of a controller;

FIG. 12 shows an exterior view of an example of a device and an exemplary flow sensor according to one embodiment;

FIG. 13 shows a cross sectional view of the example of the device and flow sensor of FIG. 12;

FIG. 14 shows an exploded view of the device and flow sensor of FIGS. 12 and 13;

FIG. 15 shows a side on cross sectional view of the device and flow sensor of FIGS. 12 and 13;

FIG. 16 is a simplified schematic diagram of a gas delivery system according to another embodiment of the present disclosure.

### DETAILED DESCRIPTION

The present disclosure relates to various systems and methods applicable to a gas delivery system arranged to deliver a breathable gas to a patient.

### Overview

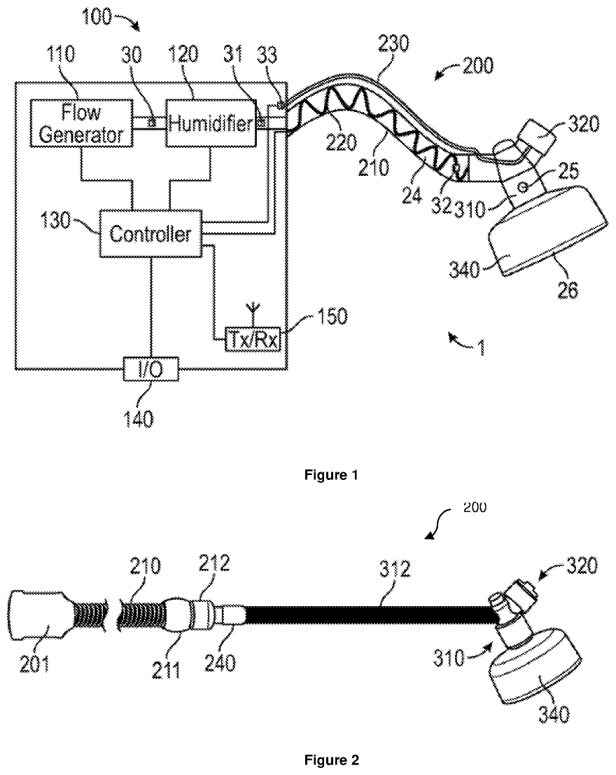

An example of a gas delivery system 1 according to one embodiment of the present disclosure is shown in FIG. 1.

The gas delivery system 1 is configured to provide respiratory therapy to a patient, by delivering a breathable gas to an airway of the patient. The respiratory therapy may be a pressure therapy delivered to a patient to assist with breathing and/or treat breathing disorders. The pressure therapy may involve the gas delivery system 1 providing pressure at, or near, the patient at one or more target pressures for one or more time windows. The pressure therapy can be infant resuscitation therapy, positive airway pressure therapy (PAP), continuous positive airway pressure therapy (CPAP), bi-level positive airway pressure therapy, non-invasive ventilation, bubble CPAP therapy or another form of pressure therapy. In some configurations, the device may provide bi-level positive airway pressure therapy to achieve infant resuscitation.

‘Pressure therapy’ as used in this disclosure may refer to delivery of a breathable gas to a patient at a pressure of greater than or equal to about 1 cmH₂O and is either delivered to mimic natural breathing cycles of a patient, and/or delivered in accordance with the patient's breathing cycles to assist with the patient's breathing.

In some configurations, the breathable gas delivered to the patient is, or comprises, oxygen. In some configurations, the gas comprises a blend of oxygen or oxygen enriched gas, and ambient air. In some configurations, the percentage of oxygen in the gases delivered may be between about 20% and about 100%, or between about 30% and about 100%, or between about 40% and about 100%, or between about 50% and about 100%, or between about 60% and about 100%, or between about 70% and about 100%, or between about 80% and about 100%, or between about 90% and about 100%, or about 100%, or 100%. In at least one configuration, the gases delivered may be of atmospheric composition. In at least one configuration, the gases delivered may be ambient air.

In relation to infant resuscitation, when in utero, the lungs of a fetus are filled with fluid, and oxygen comes from the blood vessels of the placenta. At birth, the transition to continuous postnatal respiration occurs, assisted by compression of the lungs by the birth canal. Also assisting the infant to breathe is the presence of surfactant that lines the alveoli to lower surface tension. The need for infant resuscitation can occur in a range of circumstances as will be described further below.

While most infants tolerate passage through the birth canal for the duration of the average contraction, the few that do not may require assistance to establish normal breathing at birth. Resuscitation may also be needed by babies with intrapartum evidence of significant fetal compromise, babies being delivered before 25 weeks gestation (particularly since surfactant production does not begin until the 24th week of gestation and continues until the 34th week of gestation), babies being delivered vaginally by breech, maternal infection and multiple pregnancies. Additionally, transition at birth requiring medical interventions, and especially for deliveries before 39 weeks gestation.

In general terms, the gas delivery system 1 comprises a respiratory apparatus 100, a conduit assembly 200 including one or more interconnected tubes, and an interface assembly including a patient interface 340 arranged to be in communication with an airway of a patient. In some embodiments, a device 320 fluidly couples the conduit assembly 200 to the patient interface 340. The device 320 can either be part of the patient interface 340, or it can be a device made separately from the interface 340 and later coupled to the interface 340 in use. In at least some embodiments, the device 320 includes suitable connectors, or formations, allowing it to fluidly couple to an inlet of the patient interface 340 at one end, and fluidly couple to a connector of the conduit assembly 200 at another end. In some embodiments, the device 320 and the patient interface 340 are collectively referred to as an interface assembly in this disclosure.

FIG. 2 shows an example of a conduit assembly 200 connected to an interface assembly. The conduit assembly 200 includes interconnected tubes 210, 312, which are coupled to each other via connectors 211, 212. The tube 210 may be arranged to connect to a source gas flow via another suitable connector 201.

With reference to FIG. 1 again, the respiratory apparatus 100 may include a flow generator 110, an optional humidifier 120 for humidifying the gases generated by the flow generator 110, and an associated controller 130 which manages operation of the flow generator 110 and/or the humidifier 120, when present. In at least one embodiment, the flow generator 110 can be in the form of a blower 110.

The controller 130 of the respiratory apparatus 100 may include an associated user interface 140, comprising, for example, a display and input device(s) such as button(s), touch screen, or the like. The controller 130 is configured or programmed to control and/or interact with components of the gas delivery system 1, including one or more of the following: operating the flow generator 110 to create a flow of gas for delivery to a patient, operating the humidifier 120 (if present) to humidify and/or heat the generated gas flow, receiving one or more inputs from sensors 30, 31, 32, 33 and/or the user interface 140 for reconfiguration and/or user defined operation of the gas delivery system 1, and output information to an operator on the display.

The controller 130 can control the respiratory apparatus 100 to generate a gas flow at a desired pressure, or a desired flow rate. In particular, the controller 130 controls the flow generator 110 to generate a gas flow at a desired pressure and/or flow rate.

The controller 130 may also control the humidifier 120, if present, to humidify the gas flow and/or heat the gas flow to an appropriate level. The gas flow is directed out through the conduit assembly 200 and patient interface 340 to the patient. The controller 130 can also control a humidifier heating element of the humidifier 120 and/or the heating element 220 of the conduit 210 to heat the gas to and/or maintain the gas at a desired temperature. The controller 130 can be programmed with or can determine a suitable target temperature and/or humidity of the gas flow. The controller 130 can be programmed with or can determine a suitable target temperature and/or humidity of the gas flow, and use one or more of the humidifier heating element, conduit heating element 220, and the flow generator 110 to control flow and/or pressure to the target temperature and/or humidity. The target temperature and/or humidity of the heated gas can be set to achieve a desired level of therapy and/or comfort for the patient.

Operation sensors 30, 31, 32 and 33 can be placed in various locations in the respiratory apparatus 100 and/or the conduit assembly 200 and/or patient interface 340. One or more outputs from the sensors 30, 31, 32, 33 can be monitored by the controller 130, to assist it to operate the gas delivery system 1 in a manner that provides optimal therapy. In some configurations, providing optimal therapy includes meeting a patient's inspiratory demand. In at least one configuration, providing optimal therapy includes providing a first target pressure to the patient at a first time, and a second target pressure to the patient at a second time.

The respiratory apparatus 100 may have a transmitter 150, receiver 150, and/or transceiver 150 to enable the controller 130 to receive transmitted signals from the sensors 30, 31, 32, 33 and/or to control the various components of the gas delivery system 1. The controller 130 may receive transmitted signals from the sensors related to, or control components including but not limited to the flow generator 110, humidifier 120, or humidifier heating element 220. As discussed above, the gas delivery system 1 comprises a conduit assembly 200 for receiving a breathable gas from the respiratory apparatus 100 and directing the gas flow toward the patient interface 340.

In at least one configuration, the patient interface 340 can be in the form of a sealed patient interface. In at least one configuration, the patient interface 340 can be in the form of a respiratory mask, or endotracheal tube, or laryngeal mask. The patient interface 340 can be configured to deliver a breathing gas to the patient's airway via a seal or cushion, of the patient terminal end 26, that forms an airtight seal in or around the patient's nose and/or mouth. The patient interface 340 can be an oronasal, nasal, direct nasal, and/or oral patient interface, which creates an airtight seal between the patient terminal end 26 and the nose and/or mouth of the patient. In at least one embodiment, the seal or cushion can be held in place on the patient's face by headgear. In at least one embodiment, the patient interface 340 can be held on the patient's face by an operator who may be a healthcare professional. Such sealed patient interfaces can be used to deliver pressure therapy to the patient. Alternative patient interfaces, for example those comprising nasal prongs can also be used. In some examples, the nasal prongs may be sealing or non-sealing.

A neonatal interface may be any interface, such as described above, that is configured for use with a neonate. The neonatal interface may be configured to at least partially, and preferably substantially seal around the nose and mouth of the patient.

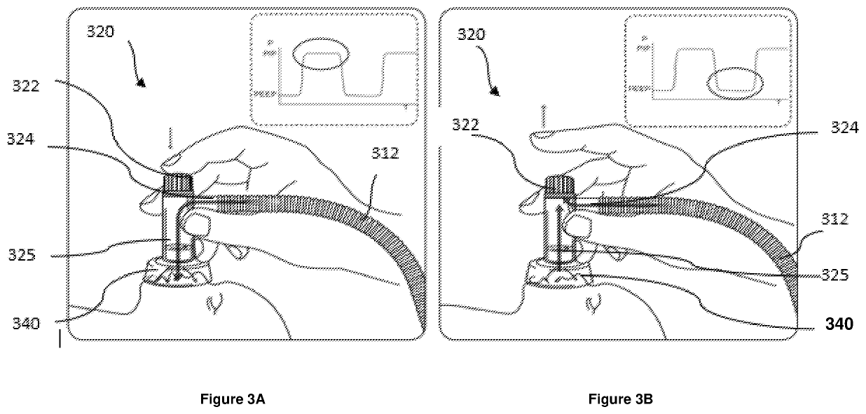

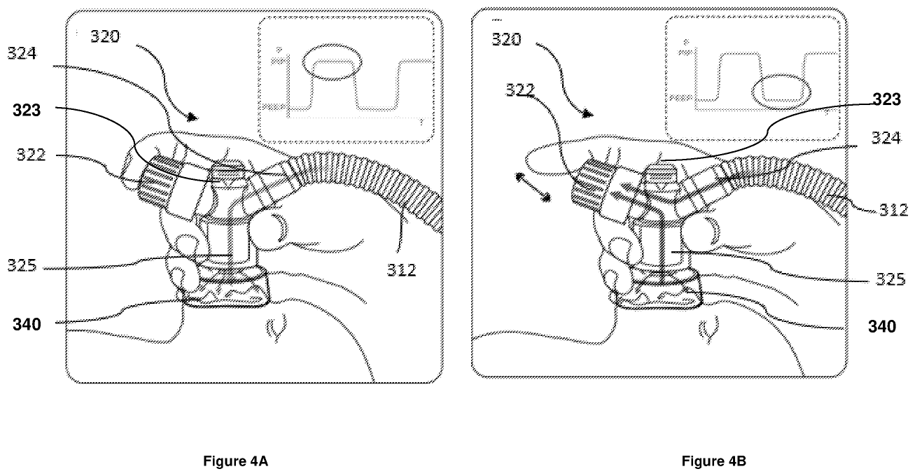

In some embodiments, a device 320 is provided for use with the gas delivery system 1, to trigger the respiratory apparatus 100 at the patient's end, that is remote from the respiratory apparatus 100, to adjust the pressure of gas delivered to the patient. That is, the triggering takes place at the patient's end via the device 320, without directly change the settings of the respiratory apparatus 100. FIGS. 3A, 3B, 4A, 4B show two types of such devices which are sometimes called T-piece devices.

As mentioned above, existing respiratory apparatuses are often configured to operate based on a fixed gas flow source which supplies a flow of breathable gas at a constant flow rate. By occluding or unoccluding a PEEP orifice of the T-piece device, different pressures of breathable gas can be provided to the patient. In addition, on existing T-piece devices both the “PIP” and “PEEP” pressures are at least partially dependent on source flow, and interface leak. This means manual adjustment of the fixed gas flow source or the respiratory apparatus may be required to keep the delivered pressures at the desired level. In some cases, the delivered pressure can be switched to a different setting due to ‘false triggers’ such as patient breathing, coughing, patient's movement, mask distension, hose flexing, and leaks. The present disclosure aims to ameliorate one or more issues faced by such systems, or to at least provide a useful alternative. According to embodiments disclosed herein, the respiratory apparatus is remotely triggered to supply a breathable gas to a patient at targeted pressure levels, by occluding or unoccluding a trigger port of a device of the respiratory system, which causes the respiratory apparatus to configure its settings.

With reference to FIGS. 3A, 3B, 4A, 4B, each device 320 includes an inlet 324 arranged to receive a breathable gas from the respiratory apparatus 100 via the conduit assembly 200, and an outlet 325 which is connected to the patient interface 340 when delivering respiratory therapy. Each device 320 also includes a trigger port 322 arranged to be occluded or unoccluded with an object, for example, a finger of an operator or a healthcare professional when delivering respiratory therapy to the patient. The trigger port 322 also forms a vent of the device 320. When the trigger port 322 is occluded, most or all of the breathable gas received from the flow generator 110 is delivered to the patient via the patient interface 340, and the gas delivery system 1 delivers the breathable gas at a first pressure to a patient. When the occlusion is removed from the trigger port 322, the trigger port 322 operates as a vent and allows some of the breathable gas to exit from an internal cavity of the device 320 to ambient air, and the gas delivery system 1 delivers the breathable gas at a second pressure to the patient. An optional valve, for example, a duckbill valve 323 may also be included in the T-piece device 320, which can be used for insertion of an auxiliary equipment such as a gas sampling device, or a catheter for fluid clearance or surfactant delivery.

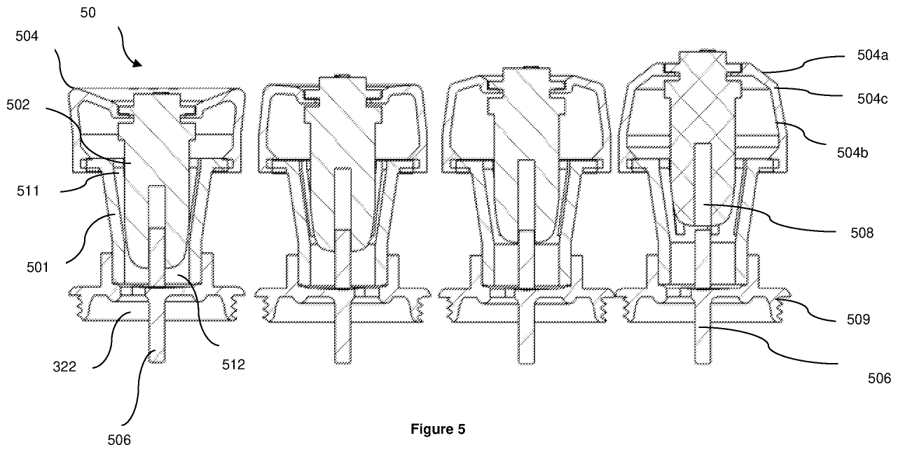

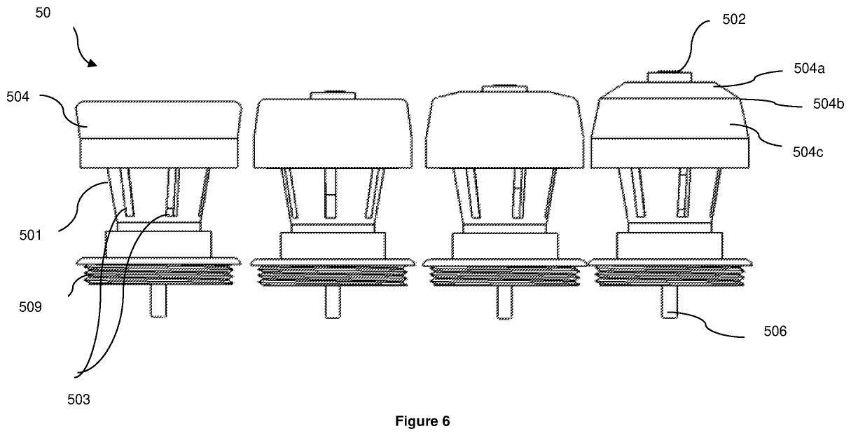

In at least some configurations, the trigger port 322 may be arranged to be occluded or unoccluded by a valve including a movable actuator, wherein movement of the actuator adjusts a flow path through the valve. An exemplary embodiment of such valve is shown in FIGS. 5 and 6. FIG. 5 illustrates various cross sectional views of the valve 50, with its actuator 502 being in a range of different operating positions, and FIG. 6 illustrates exterior side on views of the valve 50.

The valve 50 includes a housing 501, comprising a first and a second opening 512, 511, at opposite ends of the housing 501. The first opening 512 may be fluidly connected to the trigger port 322 when in use, and the second opening 511 is configured to movably receive an actuator 502. The housing 501 includes a body extending between the first and the second opening 512, 511, which forms a hollow cavity. A side wall of the body tapers from the second opening 511 to the first opening 512, such that the first opening 512 is of a smaller diameter than the second opening 511. A plurality of openings 503 are formed in the side wall of the body, as shown in FIG. 6, configured to allow gas from within the gas delivery system 1 to flow through depending on the relative position between the actuator 502 and the housing 501.

As indicated, the actuator 502 is arranged to move between a lifted and an inserted position to adjust the size of the gas flow path through the valve 50. In the lifted position, the gas is able to flow into the first opening 512 of the housing 501, and then through the plurality of the openings 503, to exit from the gas delivery system 1 to ambient air. This has an effect on lowering the pressure delivered to the patient as compared when the actuator 502 is in the inserted position. In the inserted position, the actuator 502 is lowered into the housing 501, to occlude the first opening 512 of the housing 501. This will block the air flow path through the valve 50, such that air can no longer escape the gas delivery system via the valve 50 and the trigger port 322, which elevates the pressure delivered to the patient. When the actuator 502 is in the lifted position, the pressure of the breathable gas delivered to the patient corresponds to PEEP, and when the actuator 502 is in the inserted position, the pressure of the breathable gas delivered to the patient corresponds to PIP. As the actuator 502 is repeatedly moved between these positions, pressures between PEEP and PIP are delivered to the patient.

The valve 50 may include a deformable membrane 504 which assists with the movement of the actuator 501. As shown in FIG. 5, the membrane 504 forms a chamber extending between the second opening 511 of the housing 501, and a shoulder of the actuator 502. The membrane 504 is configured such that it biases the actuator 502 in the lifted position when no external force is applied to the actuator 502. As a pressing force is applied to the actuator 502, for example, by an operator, the membrane 504 starts to deform. As the actuator 502 moves past a deflection point of the membrane 504, it moves or snaps the actuator 502 into its fully inserted position.

The mechanism that allows the membrane 504 to bias the actuator 502 in its lifted position and snaps the actuator 502 into its inserted position, is controlled by elasticity and geometry of the material used to construct the membrane 504. The membrane 504 includes a first member 504a and a second member 504b, which are joined at an angle. The joint 504c between the two members functions as a flexible hinge, allowing the relative flexing movement of the two members of the membrane. When the actuator 502 is in the lifted position and members 504a and 504b are both at rest. This is a stable and resting position for the membrane 504. As the actuator 502 is pressed down, the membrane 504 starts to stretch or deform, until it reaches a deflection point. Once the actuator 502 moves past this deflection point, due to the elasticity of the membrane, it snaps the actuator 502 into the inserted position. This is a semi-stable position for the membrane 504. As the force applied onto the actuator 502 is removed, the membrane 504 moves itself back to the stable position. The valve 50, and particularly its membrane 504 provide an improved control of the occlusion and unocclusion of the trigger port 322, by enabling the gas flow path through the valve 50 to be gradually changed, and by providing haptic feedback to an operator that is using the valve 50.

A guiding member 506 may also be provided in the valve 50, which helps to maintain the actuator 502 in an upright direction as it moves between lifted and inserted positions. FIG. 5 shows an example of such guiding member 506, formed as a vertical rod and positioned below a tapering end of the actuator 502. A receiving channel or recess 508 is formed in a lower end of the actuator 502, and receives the guiding member 506 as the actuator 502 moves into its inserted position.

Connector portions 509 may also be formed in a base of the valve 50, allowing a coupling to be made between the valve 50 and the trigger port 322.

It should be appreciated that the valve 50 illustrated in FIGS. 5 and 6 is for exemplary purpose only, to demonstrate that the occlusion and unocclusion of the trigger port 322 may be achieved via a valve 50, instead of using an operator's digit. Other examples of valves suitable for use with the trigger port 322 of a T-piece device are mentioned in US provisional application U.S. 63/269,289, the content of which is incorporated herein in its entirety by reference.

The configuration of the devices 320 in FIGS. 3A-4B allows for one handed operation during respiratory therapy, that is, by occluding or unoccluding the trigger port 322 of the device 320, such that the gas delivery system 1 delivers the breathable gas to the patient at first and second pressures to mimic breathing cycles of the patient, typically at 30-60 breaths per minute.

In at least some embodiments, the pressure of breathable gas delivered to the patient is controlled by operating the flow generator 110 of the respiratory apparatus 100 at a required motor speed. More specifically, if a higher pressure is required to be delivered to the patient, a rotational speed of the motor is increased, whereas if a lower pressure is required, the rotational speed of the motor is decreased. The motor of the flow generator 110 may be used to power a fan/impeller. Accordingly, when the motor operates at a higher speed, the blower fan also rotates at a faster speed such that a higher pressure of breathable gas is supplied to the patient and elevates the pressure inside the patient interface 340 to the required level. When the motor operates at a lower rotational speed, the blower fan is caused to rotate at a lower speed, thereby reducing the pressure of breathable gas delivered to the patient, which reduces the pressure inside the patient interface 340 to the required level.

In some embodiments, the first pressure level is delivered at or near the patient terminal end 26 at a first time or during a first time window. The first pressure level may be delivered at or near the patient terminal end 26 once interface fit is confirmed, and/or after a control signal is generated by the controller 130, which is used to configure the flow generator 110 to set its motor speed, as mentioned above.

Similarly, a second pressure level can be delivered at or near the patient terminal end 26 at a second time or during a second time window. The second pressure level may be delivered at or near the patient terminal end 26 once interface fit is confirmed, and/or after a control signal is generated by the controller 130, which is used to configure the flow generator 110 to set its motor speed. The controller 130 may try and continuously control the flow generator 110 such that the gas delivery system 1 continuously provides the breathable gas to the patient at the first and the second pressure level in order to mimic patient's breathing cycles. Typically 30-60 breathing cycles/minute are provided to the patient during respiratory therapy. In some applications, a patient's breathing cycles are manually determined by a clinician. It should be appreciated that the number of breathing cycles/minute required is largely dependent on the type of therapy to be provided to the patient, patient's condition (age, breathing condition), and may vary from patient to patient.

In at least one embodiment the first pressure level is equal to desired PIP. Preferably the second pressure is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H2O, and a useful value may be selected between any of these ranges (for example about 15 to about 60, about 15 to about 60, about 20 to about 25, about 21 to about 30, about 21 to about 27, about 21 to about 25, about 22 to about 30, about 22 to about 29, about 22 to about 25, about 23 to about 30, about 23 to about 28, about 23 to about 26, about 24 to about 30, about 24 to about 29, about 24 to about 28, about 24 to about 26 or about 25 to about 30 cm H2O). A higher PIP may be needed for first few breathing cycles (for clearing liquid from airways and beginning lung aeration) and/or if the patient does not respond positively to initially given respiratory therapy. In addition, the level of pressure required for resuscitation usually varies from patient to patient, depending on factors such as maturity of lungs, presence of lung disease, disorder, and similar. The pressure ranges mentioned above are for guide only and in practice pressures need to be individually adjusted depending on patient's response.

In one embodiment, the second pressure level is equal to desired PEEP. The second pressure may be 1, 2, 3, 4, 5, 6, 7, 8, 9, 10, 11, 12, 13, 14 or 15 cm H₂O, and a useful value may be selected between any of these ranges (for example, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 2 to about 8, about 2 to about 6, about 2 to about 5, about 3 to about 8, about 3 to about 5, about 4 to about 8, about 4 to about 7, about 4 to about 5, about 5 to about 8 or about 6 to about 8 cm H2O). The second pressure may be about 5 cm H2O, but can be set depending on, for example, patient requirements and/or clinician preference.

In some embodiments, the device 320 may be configured to be removably connected to the conduit assembly 200, and/or removably connected to the patient interface 340, for ease of replacement and cleaning after use. In some embodiments, the device 320 may be permanently connected to a conduit 312 of the conduit assembly 200, and/or permanently connected to the patient interface 340. In some embodiments, the device 320 may be an integral part of the patient interface 340.

### Method of Control

According to the present disclosure, there is provided a method of controlling a patient gas delivery system 1 described above, by: determining a source gas flow; determining a delivery gas flow; calculating a difference between the source gas flow and the delivery gas flow, and causing the gas delivery system 1 to deliver a breathable gas to the patient at a pressure based on the calculated difference between the source and delivery gas flow.

In an alternative embodiment, the present disclosure provides a method of delivering a breathable gas to a patient via an interface assembly, said interface assembly including a trigger port 322, comprising: determining a status of the trigger port 322, by determining a source gas flow generated by the gas delivery system 1 and a delivery gas flow delivered to the interface assembly, and causing the gas delivery system 1 to deliver a breathable gas to the patient at a pressure based on the determined status of the trigger port 322.

The two methods mentioned above both involve determining a source gas flow, and determining a delivery gas flow, and then using these two measurements to operate the gas delivery system 1 such that the breathable gas is delivered to the patient at a suitable pressure level.

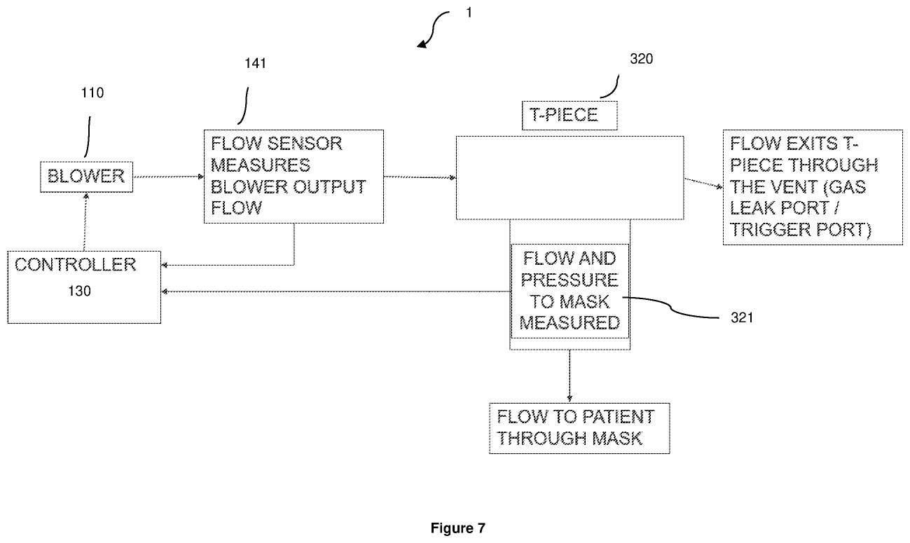

FIG. 7 illustrates a simplified schematic diagram of a gas delivery system 1 which may be used to implement one or both of the methods mentioned above. The gas delivery system 1 includes a flow generator 110, which is a blower in this example, arranged to generate the breathable gas to be delivered to the patient. For ease of illustration, the conduit assembly 200 and the patient interface 340 are both omitted from FIG. 7. The operation of the flow generator 110 is controlled by an associated controller 130. The gas delivery system 1 additionally includes a number of sensors 141, 321 arranged to measure the source gas flow, and the delivery gas flow. In one embodiment, the sensor 321 may also be used to determine a pressure of the breathable gas in the patient interface 340.

A pressure sensor may be provided to measure the pressure of the breathable gas inside the patient interface 340. The pressure sensor may be located at a suitable sensing location for it to measure the pressure of the breathable gas in the device 320 or in the patient interface 340. The pressure sensor may be a strain gauge, capacitance, electromagnetic type pressure sensor. Depending on the sensor configuration, it may be entirely located in the gas flow path and may provide its measurements back to the controller 130 via wired or wireless communications. Alternatively, a suitable pressure tapping point may be formed in the device 320 or the patient interface 340 and a component of the pressure sensor may be arranged to obtain pressure measurements via the pressure tapping point. For example, a gas bypass channel, or a pressure sensing port can be provided in the device 320 or the patient interface 340. One or more pressure sensing tubes could then establish fluid connection between the pressure tapping point and a sensing element located outside of the device 320 and the patient interface 340.

Readings of the sensors 141 and 321 are received, or calculated, by the controller 130 in order to make a determination as to the status of the trigger port 322, and what level of pressure should be delivered to the patient, and corresponding control signals are then generated and forwarded to the flow generator 110 from the controller 130, to allow it to set its motor speed.

For completeness, it is noted that it is also within the scope of the invention for the motor speed of the flow generator 110 to be used as factor in determining a current status of the trigger port 322, in addition to, or alternatively to, using the detected flow and/or pressure. By way of example, the motor speed of the flow generator 110 may be provided by an associated motor module. The controller 130 may be in signal communication with the motor module. If characteristics of the motor and other components of the system (such as the patient interface, the conduit assembly, and the trigger port) are known, then expected values (in terms of motor speed and flow, and/or motor speed and pressure) corresponding to the open and closed status of the trigger port will also be known (or able to be calculated). For instance, using this methodology, the controller can firstly determine whether the flow generator 110 is currently delivering PEEP or PIP pressure to the patient; and, based on the pressure being delivered to the patient, the controller 130 can then determine the current status of the trigger port 322.

More specifically, the source gas flow is the flow rate of breathable gas generated by the flow generator 110 and conveyed into the conduit assembly 200. This is the first flow rate determined by the controller 130, and is denoted as Fs throughout this disclosure. The first flow rate can be determined by the first sensor 141 which may be a flow sensor which is at least partially placed in a fluid path near the flow generator 110. Measurements of the flow sensor 141 are transmitted to the controller 130 via a wired or wireless sensing circuit. In one embodiment, the sensor 141 may be a pressure sensor, measurements of which can be converted to the first flow rate based on relationship of pressure and flow rate. In at least some embodiment, the pressure sensor may include one or more pressure sensing tubes that are in fluid communication with the breathable gas as it enters the conduit assembly 200. The one or more sensing tubes send a flow of the breathable gas to a corresponding sensing element in the controller 130, at which point determination of the flow rate is then carried out.

The delivery gas flow refers to the flow rate of breathable gas delivered to the patient interface 340. This is the second flow rate determined by the controller 130, and is denoted Fm throughout this disclosure. Fm includes the breathable gas that the patient inhales during respiratory therapy, but it also includes any interface leak (if there is any). The difference between Fs and Fm is independent of interface leak and other sources of system noise (such as patient coughing), and is representative of the breathable gas flow through the trigger port 322. As such, this difference (i.e. Fs−Fm) should be small, when the trigger port 322 is occluded, that is, in the closed state, and the difference should be at a higher value which approximately equals the amount of breathable gas flows through the trigger port 322, when the trigger port 322 is unoccluded (in the open state). The controller 130 determines or calculates this difference (Fs−Fm), and then uses the difference to determine which state the trigger port 322 is currently in, or whether the trigger port 322 status has changed.

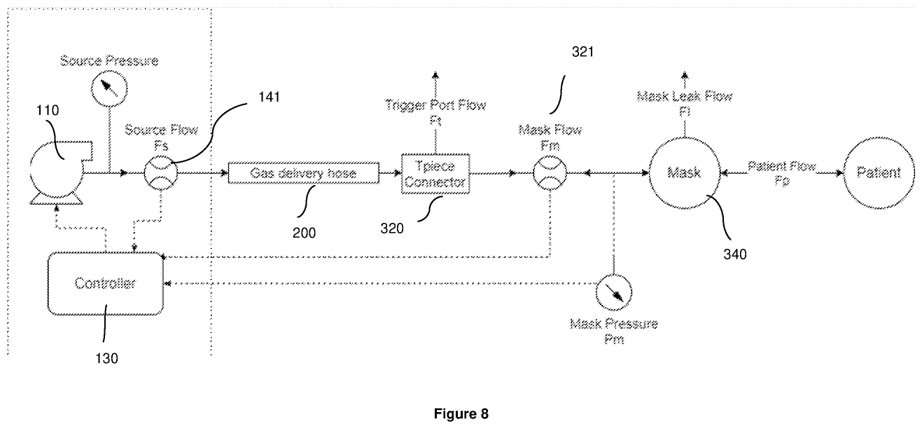

FIG. 8 is a schematic diagram of the gas delivery system 1, including arrows which indicate direction of gas flows in the gas delivery system 1, and the relevant flow rates used by the controller 130 to implement its control function of the flow generator 110. As illustrated, the source gas flow Fs, that is, the flow rate of breathable gas generated by the flow generator 110 and conveyed into the conduit assembly 200, is measured based on flow characteristics of the breathable gas near the flow generator 110 end. In comparison, the delivery gas flow Fm, that is, the flow rate of breathable gas delivered to the patient's interface 340, is measured based on flow characteristics of the breathable gas near the patient's end, and preferably, at or near the device 320 itself. FIG. 8 also shows two pressure measurements, namely source pressure Ps and mask pressure Pm (or interface pressure). These pressure measurements may be carried out by the first and second sensors 141, 321 directly as will be further described below, or they can be measured by additional pressure sensors.

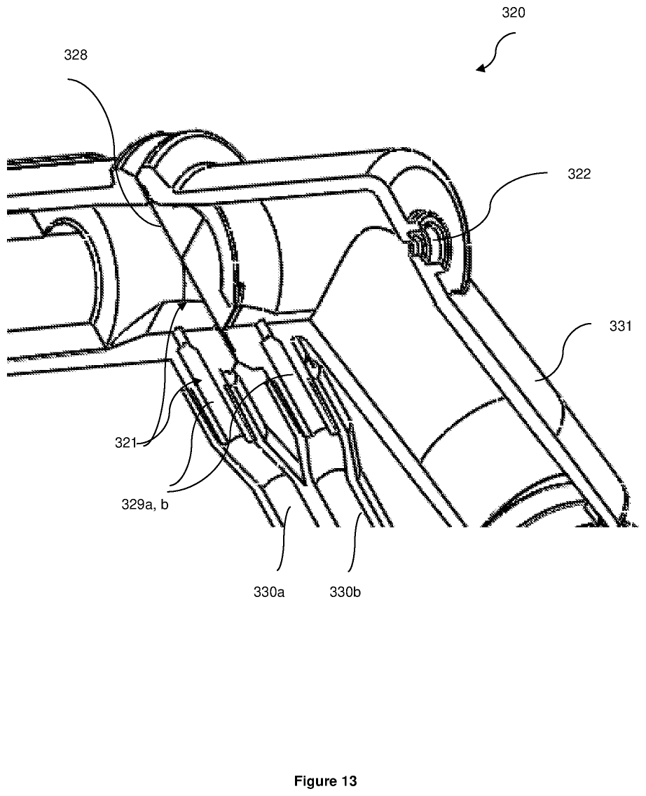

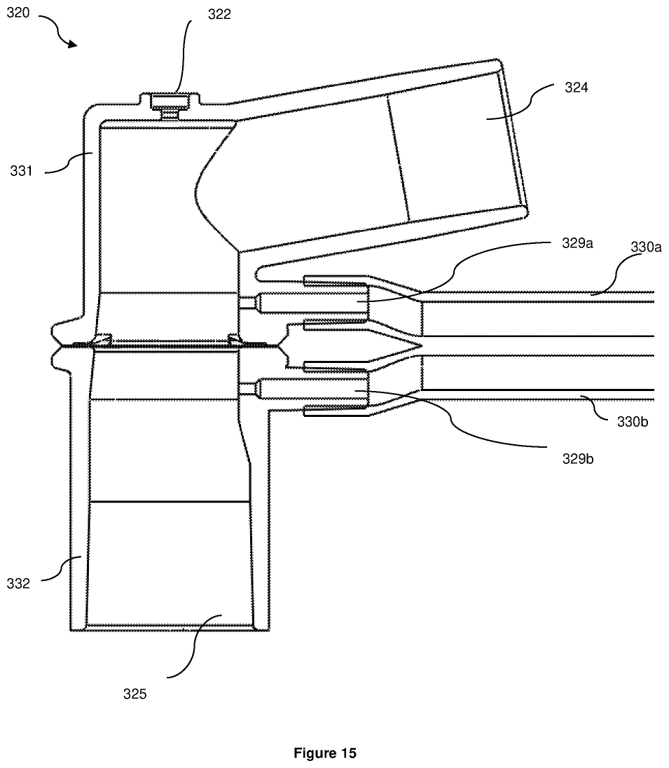

Similar to the first sensor 141 described above, the second sensor 321 may be an integrated sensor, measurements taken by the sensor 321 are transmitted back to the controller 130 either via a wired circuit or via wireless signal transmission. In some embodiments, the second sensor 321 may be configured such that it comprises a sensing component (for example, a flow restriction such as a diaphragm or a deflectable flap) placed in a gas flow path or in a pressure tapping position, and pneumatic connections established by one or more pressure sensing tubes located outside of the cavity of the device 320 and the patient interface 340. This will be described later with reference to FIGS. 12 to 16, which show one exemplary embodiment of a device 320 including a differential pressure sensor.

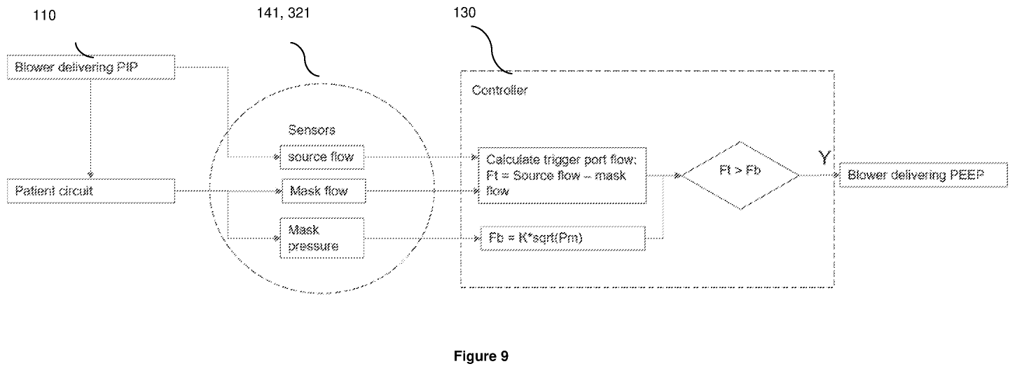

FIGS. 9-11 illustrate examples of control logic implemented by the controller 130 to determine whether the trigger port 322 is in an open or closed state, which is then used by the controller 130 to operate the flow generator 110 to deliver a target pressure to the patient. In the examples shown, the flow generator 110 is operated by the controller 130 such that it delivers the breathable gas to the patient repeatedly at a first pressure level, and a second pressure level to mimic patient's breathing cycles. Preferably, the first pressure is higher than the second pressure, and more preferably, the first pressure corresponds to PIP, whereas the second pressure corresponds to PEEP.

For completeness, it is noted that it is within the scope of the invention for the system to, instead of operating only in a pressure-based mode, instead operate in a “hybrid” mode wherein, depending on the status of the trigger port 322, the controller 130 may be in either a pressure-based or a flow-based mode. For instance, when the trigger port 322 is in an open state, the device may operate in a flow-based (or “flow control”) mode whereby a target flow rate(s) is aimed for. When the trigger port is in a closed state, the device may operate in a pressure-based (or “pressure control”) mode whereby a target pressure(s) is aimed for.

In some embodiments, a previous status of the trigger port 322 is stored in a memory of the controller 130. Measurements from the sensors 141, 321 are obtained or received by the controller 130, preferably at predefined frequencies. After the controller 130 calculates the difference between the first and second flow rates, it compares the difference with a first and/or a second threshold flow rates (Fa, Fb), in order to determine a current status of the trigger port 322, and then decides whether the status of the trigger port 322 has changed, by making a comparison of the current status and the previous status of the trigger port 322. There are a number of different ways to determine Fa, or Fb as will be described below.

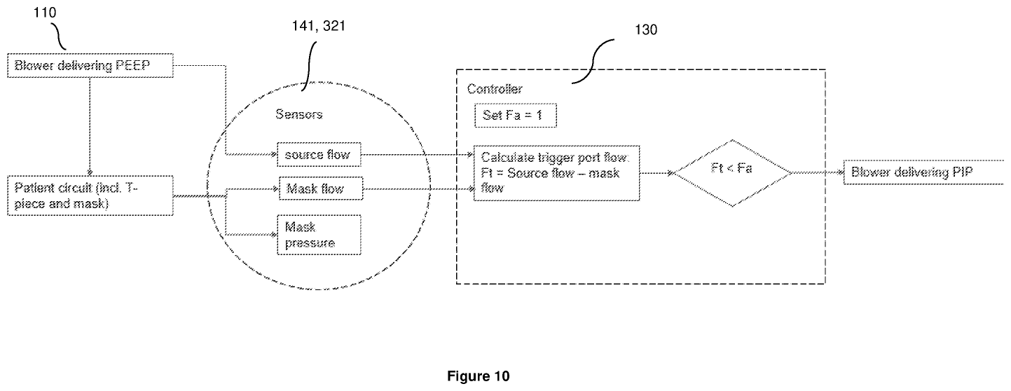

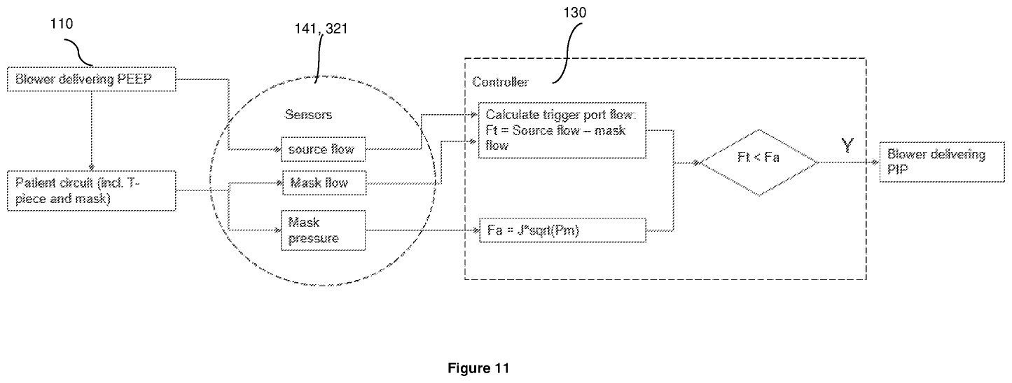

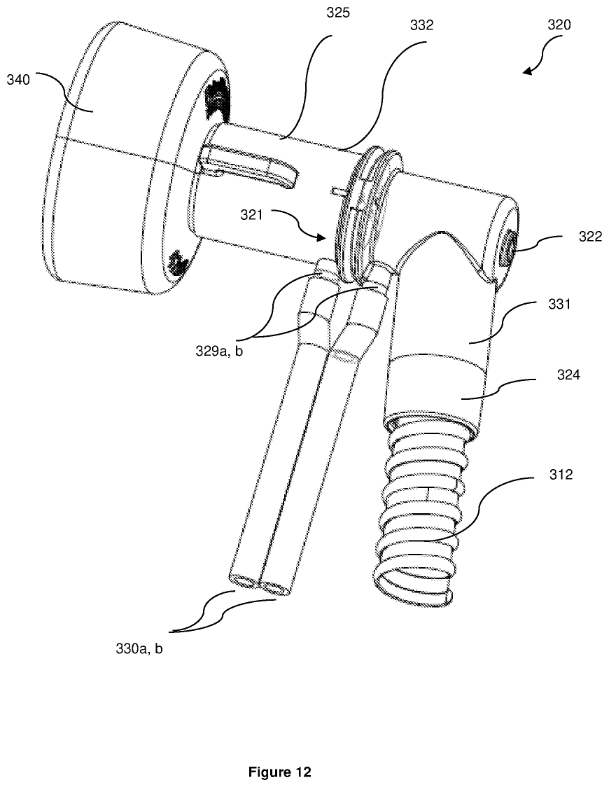

In some configurations, the first threshold flow rate is determined based on interface pressure. In some configurations, the first threshold flow rate Fa is calculated from J*sqrt(Pm), as illustrated in FIG. 11, wherein Pm is the interface pressure, and J is a coefficient determined experimentally or during a calibration stage, and/or obtained from a database or lookup table incorporated in or accessible to the system. Interface pressure Pm is the pressure of the breathable gas within the patient interface 340, which may be measured by the second sensor 321, or another pressure sensor. Alternatively, the first threshold flow rate Fa may be a predetermined range, such as between 0.5 to 10 L/min. Alternatively, the first threshold flow rate Fa may be set at a constant level/flow rate. In some configurations, the first threshold flow rate is set at a constant flow rate of 1 L/min, as indicated in FIG. 10. In at least some configurations, the coefficient J could be for instance, between 2 and 6 L/min*cmH2O^{-1/2}. In at least one embodiment, the coefficient J could be for instance, 4.47 L/min*cmH₂O^{-1/2}, which results in a threshold Fa of 10 L/min for a Pm of 5 cmH₂O.

In some configurations, the second threshold flow rate is calculated based on interface pressure Pm. In some configurations, the threshold flow rate Fb is calculated from K*sqrt(Pm), as illustrated in FIG. 9, wherein Pm is interface pressure, and K is a coefficient determined experimentally or during a calibration stage and/or obtained from a database or lookup table incorporated in or accessible to the system. Alternatively, the second threshold flow rate Fb may be a predetermined range, such as between 0.5 to 10 L/min. Alternatively, the second threshold flow rate may be set at a constant level/flow rate. In some configurations, the second threshold flow rate is set at a value between a range of 1 to 3 L/min. In at least some configurations, the coefficient K could be for instance, between 1 and 4 L/min*cmH2O^{-1/2}. In at least one embodiment, the coefficient K could be for instance, 2.23 L/min*cmH₂O^{-1/2}, which results in a threshold Fb of 5 L/min for a Pm of 5 cmH₂O.

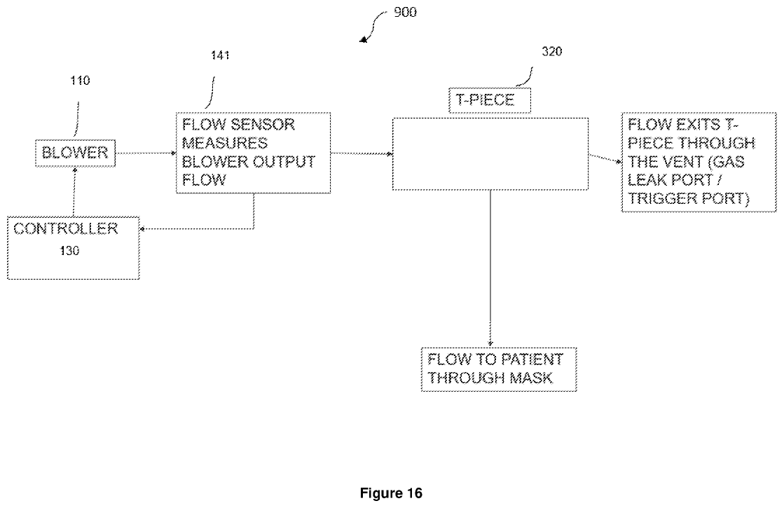

As discussed further below with reference to the alternative embodiment of FIG. 16, the coefficients J and K may be based on calculated conductance values (Ctrig, Cbias) associated with a theoretical “trigger orifice” and “bias orifice”. These theoretical orifices are mathematical representations of flow egress points/scenarios in the gas delivery system 1. The trigger orifice represents flow egress through the trigger port 322. The bias orifice represents a minimum expected flow through the gas delivery system 1 with the trigger port 322 closed and no flow egress through the patient interface 340.

Exemplarily, the coefficients J and K may each correspond to (Ctrig+Cbias). That is, the values of J and K may be each equal to Ctrig+Cbias.

In some embodiments, the first or second threshold flow rates Fa and Fb may be equal. In some embodiments, the value of Fb may be chosen to be slightly greater than Fa to account for the fact that some air may leak out from the trigger port 322 when it is in the closed state.

With reference to FIG. 9, when the flow generator 110 is already delivering PIP, the trigger port 322's previous status will be closed. The ‘previous status’ refers to the last status as recorded by the controller 130. In order to remotely trigger the flow generator 110 to switch to PEEP, the operator unoccludes the trigger port 322 (for example by removing the finger from the trigger port 322, or by using the valve 50 shown in FIGS. 5-6), which causes the breathable gas to flow through the trigger port 322 to exit to ambient air. The controller 130 compares the calculated difference of Fs and Fm, with the threshold flow rate Fb. If the difference is greater than Fb, the controller 130 makes a determination that the trigger port 322 has now changed from the closed state to the open state, and sends a control signal to the flow generator 130 to configure its motor speed such that the delivered pressure is lowered to PEEP level. The flow generator 110 is maintained at this motor speed until a new control signal is received from the controller 130.

Similarly, with reference to FIGS. 10 and 11, when the flow generator 110 is already delivering PEEP, the previous status of the trigger port 322 will be open. That is, the last recorded status of the trigger port 322, by the controller 130 is open. In order to remotely trigger the flow generator 110 to switch to PIP, the operator occludes the trigger port 322, for example by putting a finger in front of it, such that less breathable gas leaks out to ambient air via the trigger port 322. The controller 130 compares the calculated difference of Fs and Fm, with the threshold flow rate Fa. If the difference is smaller than Fa, the controller 130 makes a determination that the trigger port 322 has now changed to a closed state, and generates a control signal for the flow generator 110 to increase its motor speed such that the delivered pressure is increased to PIP level. The flow generator 110 is maintained at this level until a new control signal is again received from the controller 130.

In one embodiment, if the controller 130 decides that the current status of the trigger port 322 has changed from its previous status, the controller 130 is configured to change the pressure between PIP and PEEP over a predetermined time interval to achieve adequate breath rate or minute ventilation (delivered breaths per minute). In one embodiment, the predetermined time interval is between 100 to 400 ms, or between 100 to 300 ms, or between 100 to 200 ms, or approximately 150 ms.

The control logic described above is preferably implemented in the controller 130 by a suitable software algorithm. Upon or after receiving the measurements from the sensors 141, 321, the controller 130 may additionally perform an appropriate filtering, or smoothing calculation to the measurements, to remove any noises or false signals.

As mentioned above, the controller 130 is arranged to determine source gas flow Fs and delivery gas flow Fm based on measurements provided by sensors 141 and 321. These sensors can be in various different forms, such as mass flow sensors, pressure sensors, differential pressure sensors, flow rate sensors, ultrasonic flow sensors, and/or thermistors. In some embodiments, one or both of the sensors 141, 321 comprise pressure sensors, the pressure measurements of the which may be used by the controller 130 to calculate the first and second flow rates mentioned above.

In at least some embodiments, the controller 130 and the flow generator 110 may be embedded in a respiratory apparatus 100, which receives a source of breathable gas, for example from a fixed wall source, when delivering respiratory therapy to a patient. The sensors 141 and 321 may be provided at suitable sensing locations outside an enclosure of the respiratory apparatus 100. In the case of sensors which can obtain measurements via one or more pressure tapping points such as a bypass channel or sensing tubes, they may be accommodated in the enclosure of the respiratory apparatus 100.

In at least some embodiments, one or more of the sensors 141, 321 may be integrated sensors, allowing them to be accommodated entirely within a gas flow path of the gas delivery system 1, and their measurement signals may be transmitted back to the controller 130 via wireless or wired connections.

In another embodiment, when at least one of the one or more sensors include a pneumatic sensor, one or more sensing ports may be required in a suitable location of the T-piece device 320 to allow measurements of the gas flow. The one or more sensing ports are arranged to be fluidly connected to one or more components of the sensors, such as flaps, diaphragms, thermistors, and some components of the sensors may be located outside the cavity formed by the T-piece device 320.

Regardless of which type of sensors are used at the patient's end, as long as they can be used to determine a flow rate of breathable gas delivered to the patient interface 340, using one or more of the control methods described above, they would be suitable for the systems and methods described herein.

FIGS. 12-15 illustrate various different views of a device 320 and a patient interface 340 incorporating an exemplary sensor arrangement.

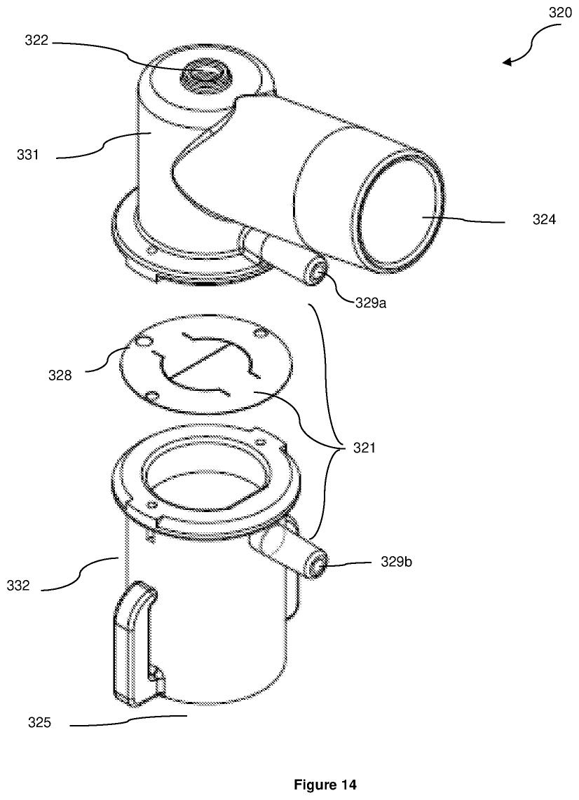

The device 320 includes an inlet 324 for receiving a breathable gas from the conduit assembly 200, an outlet 325 arranged to be fluidly coupled to the patient interface 340 when in use, and a trigger port 322. A substantially hollow body portion of the device 320 extends between the inlet 324 and the outlet 325. The body portion includes a first member 331, which includes a trigger port 322. In this particular embodiment the body portion includes a bend along the length of the body portion, forming an angle and the trigger port 322 is located on the angle. In other embodiments the body portion may be configured without the bend, for example the body portion is formed as a substantially straight hollow cavity without a bend along its length, and the trigger port 322 can be located in a side wall of the body portion or at an end of the body portion. A second member 332 of the body portion is of a substantially hollow configuration and is arranged to be connected to the patient interface 340 when in use. The first and second members 331, 332 may be joined permanently by ultrasonic welding, adhesive, or overmolding, or they can be removably coupled to each other. FIG. 14 shows an exploded view of the first member 331 and second member 332 of the device 320.

The sensor 321 may be a differential pressure sensor which has a flow restriction. The flow restriction creates a pressure drop along the gas flow path, used to derive the flow rate of the gas flowing through the device 320. For example, the flow restriction may be, or may comprise, an orifice plate or a deflectable flap. In the example shown in FIGS. 12-15, the sensor 321 is a differential pressure sensor which includes a deflectable flap 328 positioned between the first and members 331, 332 described above. The deflectable flap 328 acts as a variable flow restriction for the gas that passes through the internal cavity of the device 320 and creates a difference in gas pressures on either side of the deflectable flap 328, which is used to derive the flow rate of the gas flowing through the device 320 by the controller 130 (i.e. the deflection of the flap 328 is dependent on the pressure differential on either side of the flap 328). It should be appreciated that sensors can come in many different forms, some may require a plurality of such deflectable flaps 328. In addition, in some sensors the deflectable flap 328 can be attached to a variety of angular or displacement sensors e.g. rotary encoder, strain gauge, hall effect, and similar thereof. Differential pressure or flow rate measurements can then be derived based on readings of the angular or displacement sensors.

With reference to FIGS. 12-15 again, a first and a second pressure sensing ports 329a, 329b can be formed in a side wall of the device 320. The pressure sensing ports 329a and 329b are pressure tapping points that create a fluid path between an internal cavity of the device 320, on either side of the deflectable flap 328, and other components of the differential pressure sensor which are located externally to the device 320. In use, a pair of sensing tubes 330a, 330b are fitted over the pressure sensing ports 329a, b respectively. In equilibrium, the pressure of air within the two sensing tubes 330a, 330b will equal the pressure of air in the device 320 on either side of the flap 328. In a simplest form, the pressure sensing tubes 330a, 330b may be two flexible tubes which form an entirely pneumatic pressure connection between the device 320, and a sensing element provided in the controller 130. The controller 130 is programmed to calculate a bi-directional flow rate based on differential pressure measurements. A pneumatic pressure sensor eliminates the need of providing electrical circuit connections between the device 320 and the controller 130 and may be preferred in some instances.

For completeness, the following additional points are noted in relation to embodiments comprising one or more pressure tapping points/pressure sensing ports and one or more pressure sensing tubes:
In some embodiments, the pressure tapping points/pressure sensing ports 329a, 329b may be both located on one of the members 331, 332 of the body portion. For instance, they may be located on the first member 331 of the body portion, such that they (and the one or more pressure sensing tubes) can be attached and detached together with the trigger port 322.
More generally, in some embodiments the one or more pressure sensing tubes 330a, 330b may be removably attached to the body portion. They may be removable in their own right, and/or they may be attached to a component of the body portion that is itself removable/detachable from the other components of the body portion.
In some embodiments, the system may have a “backup” protocol in place for determining pressure, if for instance there is a blockage affecting the pressure tapping points/pressure sensing ports and/or the one or more pressure sensing tubes. For instance, the “back-up” may comprise estimating pressure at the patient interface based on flow. Alternatively or additionally, the system may comprise additional pressure sensors elsewhere in the system to be used as a backup.

### ALTERNATIVE EMBODIMENT

FIG. 16 shows a gas delivery system 900 according to another exemplary embodiment of the present disclosure.

In this embodiment, the gas delivery system 900 is configured with one or more sensors 141 proximate the flow generator 110; however, in this embodiment there are no sensors proximate the patient interface 340 (not shown in FIG. 16). That is, the sensor 321 which is placed near the patient's end is omitted in this embodiment. The system 900 so configured is still able to determine whether a detected level of flow corresponds to the trigger port 322 (not shown in FIG. 16) being in an open or closed state, and to operate the flow generator 110 (via the controller 130) to control the pressure of breathable gas delivered to the patient interface 340 accordingly.

Specifically, in such an embodiment the one or more sensors 141 comprise at least a flow sensor proximate the flow generator 110, such that a source gas flow (Fs) can be determined.

The one or more sensors 141 may also comprise a pressure sensor proximate the flow generator 110, to measure source pressure (Ps), that is to say, pressure proximate the flow generator 110. Alternatively, the pressure (P) proximate the flow generator 110, or at any point in the system 900, may be mathematically determined based on flow (if a flow sensor instead of a pressure sensor is deployed).

Within the system 900, there are a number of flow egress (or “leak”) points/scenarios, for example, the patient interface 340, the trigger port 322, and a minimum expected flow through the system 900 with the trigger port closed and no flow egress through the patient interface (i.e. a minimum flow through the system to ensure safe clearance of gases). Each of these can be mathematically approximated by an orifice (not shown in FIG. 16) respectively: a patient interface orifice, a trigger orifice, and a bias orifice. Each of these theoretical orifices has associated with it a conductance value (conductance being a function of the flow that passes through the orifice for a given pressure). The conductance value for each of these theoretical orifices representing flow egress points/scenarios can be mathematically determined (such as determined experimentally or during a calibration stage, and/or obtained from a database or lookup table incorporated in or accessible to the system); and, once determined, the conductance values are known properties of the system.

From this, a dynamic threshold flow rate (Fth) can be determined.

Fth may be related to L*sqrt (P), with P being the mathematically-calculated pressure at the patient interface 340 based on flow, and L being a coefficient determined experimentally or during a calibration stage, and/or obtained from a database or lookup table incorporated in or accessible to the system. In at least some configurations, the coefficient L could be for instance, between 2 and 10 L/min*cmH₂O^{-1/2}. In at least one embodiment, L could be for instance, 4.47 L/min*cmH₂O^{-1/2}, which results in a threshold Fth=10 L/min for a P at the interface of 5 cmH₂O.

In one embodiment, the coefficient L is provided by (Cbias+Ctrig), being the calculated conductance values of the bias orifice and the trigger orifice. Cbias could be for instance, 2.23 L/min*cmH₂O^{-1/2}, which corresponds to a bias orifice where the bias flow is 5 L/min at a calculated mask pressure of 5 cmH₂O (according to the equation Cbias=FlowBias/sqrt(P). Ctrig could be for instance, 2.23 L/min*cmH₂O^{-1/2}, which corresponds to a trigger orifice where the flow passing through the trigger port 322 is 5 L/min at a calculated mask pressure of 5 cmH₂O (according to the equation Ctrig=FlowTrig/sqrt(P)).

In another embodiment, the coefficient L may be provided by (Cbias+CmaskMaxPIP). CmaskMaxPIP is a calculated conductance value associated with an orifice representing maximum flow egress through the patient interface during delivery of PIP. In at least one embodiment, CmaskMaxPIP could be for instance, 11.18 L/min*cmH₂O^{-1/2}, which corresponds to a hypothetical orifice passing a maximum flow (e.g., 50 L/min) at the PIP pressure (e.g., 20 cmH2O) (according to the equation CmaskMaxPIP=FlowmaskMaxPIP/sqrt(PIP)).

In an alternative embodiment, the dynamic threshold flow rate (Fth) formula may also have a component accounting for flow entering/leaving the patient's lungs (i.e. the breathable gas that the patient inhales during respiratory therapy). This may be derived from the measured or approximated lung compliance of the patient. For instance, the typical lung compliance of the type of patient being treated may be used as an approximation.

Thus, exemplarily, the dynamic threshold flow rate (Fth) equation may be as follows:

$$Fth = L * sqrt ( P ) + Fpatient ( t )$$

where Fpatient(t) is the flow entering/exiting the patient's lungs at the point in time being considered.

The source gas flow (Fs) can then be compared to the dynamic threshold Fth to determine if the trigger port 322 is in an open or closed state.

If Fs<Fth, i.e. if the source gas flow Fs is less than the dynamic threshold flow Fth, this indicates that the trigger port 322 is in a closed state.

If Fs>Fth, i.e. if the source gas flow Fs is greater than the dynamic threshold flow Fth, this indicates that the trigger port 322 is in an open state.

Optionally, as part of determining if the trigger port 322 is in an open or closed state, the system 900 may also implement a “hysteresis” analysis, which acts to stabilize the system by guarding against undesirable oscillations between the open and closed state of the trigger port 322 based on minor or borderline flow fluctuations. If the determination includes a hysteresis analysis, then as part of the determination, the system 900 will further need to determine the prior (existing) state of the trigger port 322 immediately prior to the comparison of Fs and Fth.

In other respects, the system of this embodiment may be substantially as described above with respect to other embodiments.

### Additional Functionality

In both the dual-position sensor (141, 321) and the single-position sensor (141) embodiments (as shown in, respectively, FIGS. 7 and 16, the system (1, 900) may be further configured to detect one or more of:
that the patient interface 340 is disconnected (off), or incompletely, or improperly connected;
that other components of the system (notably the humidifier (120 in FIG. 1) and/or conduit (210 in FIG. 1) are disconnected; and/or
blockage in, or of, the system 1/900.

If such a condition(s) is detected, the system may be configured to undertake one or more actions in response, for safety and/or functionality reasons.

### Detection of Patient Interface Disconnection

Patient interface disconnection will cause excessive leak/flow egress from the system (i.e. greater leak/flow egress than when the patient interface is correctly fitted).

This may be modelled by treating excessive leak/flow egress as an additional theoretical orifice of the system. This theoretical orifice will have a mathematically-determinable conductance value (Cmaskleak), being a function of the flow that passes through this theoretical orifice for a given pressure. In at least one embodiment, Cmaskleak could be for instance, 6.71 L/min*cmH₂O^{-1/2}, which corresponds to a hypothetical mask leak orifice where the flow passing through the orifice is 15 L/min at a pressure of 5 cmH₂O (according to the equation Cmaskleak=FlowMaskLeak/sqrt(P)).

From this, an excessive flow threshold (Fₗₑₐₖₜₕ) can be determined as, exemplarily:
For the single-sensor embodiment:

$${F}_{leakth} = ( Cbias + Ctrig + Cmaskleak ) * sqrt ( P ) + Fpatient ( t )$$

For the dual-sensor embodiment (sensor at source and at interface):

$${F}_{leakth} = Cmaskleak * srt ( Pm ) + Fpatient ( t )$$

In at least one embodiment, the leak threshold (Fₗₑₐₖₜₕ) can be, for instance, 80 L/min, which means that excessive leak is detected if the flow passing through the orifice is greater than 80 L/min. Similarly, if the flow passing through the orifice is lower than 80 L/min, there is no excessive leak.

The actual flow in the system can then be compared against this threshold (Fₗₑₐₖₜₕ), to determine whether or not excessive leak is present, indicating patient interface disconnection.
For the single-sensor embodiment, excessive leak is detected if:
   Fs>Fₗₑₐₖₜₕ
And cessation of excessive leak is detected once:
   Fs<Fₗₑₐₖₜₕ
For the dual-sensor embodiment, excessive leak is detected if:
   Fm>Fₗₑₐₖₜₕ
And cessation of excessive leak is detected once:
   Fm<Fₗₑₐₖₜₕ

Similar methodology can be used to detect if the patient interface is incompletely or improperly connected (which will lead to greater than expected leak, though smaller than if the patient interface is off completely).

If excessive leak is detected, indicating the patient interface is disconnected (or incompletely/improperly connected), the system may, exemplarily, set the flow to a fixed flow rate; set the motor to a fixed motor speed; or set the pressure to a fixed pressure. These actions may help to ensure the system still provides some level of respiratory support if, say, the patient interface is in on the patient's face but improperly/incompletely connected. The system may also disable gas delivery, or some gas delivery functionalities, when excessive leak is detected; such as for safety reasons. For instance, delivery at a particular pressure based on detected status of the trigger port may be disabled. The system may also generate one or more alarms or alerts, to ensure a caregiver, doctor or other party is alerted to the issue.

### Detection of Disconnection of Other Components

Disconnected components can be conceptualized as an extreme case of excessive leak. Thus, similarly to detection of patient interface disconnection, disconnected components (such as the humidifier 120 or conduit 210) can be approximated as an additional theoretical orifice of the system, whose conductance value (Cdisconnectedcomponent) can be mathematically determined.

The overall conductance value of the system (Csystem) can be determined as a function of Fs and P—where in this case P is the source pressure, i.e. pressure proximate the flow generator. Specifically:

$$Csystem = Fs / sqrtP$$

Thus, detection of the disconnected component can be determined if:
Csystem>Cdisconnectedcomponent.

System responses to detection of a disconnected component(s) can include setting a fixed motor speed (such as to a minimum speed), as well as generation of alarms or alerts.

In at least one embodiment, Cdisconnectedcomponent could be for instance, 70.71 L/min*cmH₂O^{-1/2}, which corresponds to a hypothetical orifice where the flow passing through the orifice is 100 L/min at a pressure of 2 cmH₂O (according to the equation Cdisconnectedcomponent=FlowDisconnectedcomponent/sqrt(P).

### Detection of Blockage

Blockage detection in the system can be detected by comparing actual flow with an appropriate threshold flow value. For both the dual-position sensor (141, 321) and the single-position sensor (141) embodiments of the system (1, 900) of the invention, if actual flow is lesser than the threshold flow value, it may indicate the presence of blockage of the system.

In the single-position sensor embodiment, the threshold flow value may correspond to the above-discussed minimum expected flow through the system 900 with the trigger port closed and no flow egress through the patient interface. If the actual source flow rate (Fs) is lesser than this, this indicates blockage.

In the dual-position sensor embodiment, the threshold flow value may be derived from the minimum expected flow through the system with the trigger port closed and no other flow egress flow egress through the patient interface, more particularly from the associated conductance value Cbias, in combination with interface pressure. This threshold flow value may be compared against Fs, the source flow rate. If Fs is lesser than the threshold flow value, this indicates blockage.

Optionally, lung compliance (actual or approximated) of the patient may also be included as a factor when determining the threshold flow value for the dual-position sensor embodiment.

System responses to detection of a blockage can include setting a fixed motor speed (such as to a minimum speed), as well as generation of alarms or alerts.

### Terminology

Positive End Expiratory Pressure (PEEP) is also known as Peak End Expiratory Pressure and the two terms are often used interchangeably in the context of respiratory therapy systems and methods.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes, or tends toward, a particular value, amount, or characteristic. For example, as the context may dictate, the term “generally linear” can mean something that departs from exactly parallel by less than or equal to 15°.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

Reference to any prior art in this specification is not, and should not be taken as, an acknowledgement or any form of suggestion that that prior art forms part of the common general knowledge in the field of endeavor in any country in the world. Furthermore, any references to other patent specifications, other external documents, or other sources of information are generally for the purpose of providing a context for discussing the features of certain embodiments. Unless specifically stated otherwise, reference to such external documents is not to be construed as an admission that such documents, or such sources of information, in any jurisdiction, are prior art, or form part of the common general knowledge in the art.

Embodiments of the invention may also be said broadly to be embodied in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Where, in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims that follow.

1. A device arranged to be in fluid communication with a patient interface arranged to be in communication with an airway of a patient during respiratory therapy, the device comprising:
an inlet for receiving a breathable gas from a respiratory apparatus;
an outlet for conveying the breathable gas to the patient interface;
a body portion extending between the inlet and the outlet, wherein the body portion includes a trigger port, and
one or more sensors, wherein output of the one or more sensors is used to determine a status of the trigger port.

2. The device of claim 1, wherein the body portion includes one or more sensing ports.

3. A device arranged to be in fluid communication with a patient interface arranged to be in communication with an airway of a patient during respiratory therapy, the device comprising:
an inlet for receiving a breathable gas from a respiratory apparatus;
an outlet for conveying the breathable gas to the patient interface, and
a body portion extending between the inlet and the outlet, wherein the body portion includes a trigger port, and one or more sensing ports, arranged to be fluidly connected to one or more sensors, wherein output of the one or more sensors is used to determine a status of the trigger port.

4. The device of any one of claims 1 to 3, wherein the body portion includes a first member, and the trigger port is located in a wall or an end of the first member.

5. The device of claim 4, wherein the body portion includes a second member, which is of a substantially hollow configuration creating a passage for the breathable gas to flow through.

6. The device of claim 5, wherein the second member is configured to allow attachment to an inlet of a patient interface.

7. The device of either one of claims 5 to 6 wherein the first and the second members are joined together permanently.

8. The device of claim 7, wherein the first and second members are joined together using ultrasonic welding, adhesive, or overmolding.

9. The device of either one of claims 5 to 6, wherein the first and second members are removably joined together.

10. The device of any one of claims 1 to 9, wherein the one or more sensors include one or more pressure and/or flow rate sensors.

11. The device of any one of claims 1 to 10, wherein the one or more sensors include at least one differential pressure sensor.

12. The device of claim 11, wherein the at least one differential pressure sensor includes a flow restriction in a gas flow path, wherein the flow restriction is arranged to create a differential pressure on either side of the flow restriction.

13. The device of claim 12, wherein the flow restriction may include one or more deflectable flaps, a variable orifice membrane/diaphragm, and similar there of.

14. The device of any one of claims 5 to 9, wherein a deflectable flap is positioned between the first and second members.

15. The device of any one of claims 1 to 14, wherein the one or more sensors at least partially extend into an internal cavity of the body portion.

16. The device of claim 2 or 3, wherein the one or more sensors at least partially extend into the one or more sensing ports.

17. The device of claim 1, wherein at least one the one or more sensors are accommodated within the body portion of the device.

18. The device of any one of claim 1 or 17, wherein at least one of the one or more sensors are located outside the body portion, and is arranged to receive a flow of the breathable gas from the one or more sensing ports.

19. The device of any one of claim 2 or 3, wherein the one or more sensing ports include two pressure sensing ports, which extend outwardly from a wall of the body portion.

20. The device of any one of claims 1 to 19, wherein at least one of the one or more sensors is used to measure a flow rate of breathable gas delivered to the patient.

21. The device of any one of claims 1 to 20, wherein at least one of the one or more sensors is used to measure a pressure of the breathable gas in the device, or in the patient interface.

22. The device of any one of claims 1 to 21, wherein the trigger port includes an open state, and a closed state.

23. The device of claim 22, wherein the trigger port is in a closed state if it is blocked by an object.

24. The device of claim 22 or 23, wherein the trigger port is in an open state if it is not blocked by an object, such that an airflow within the device is allowed to exit to ambient air.

25. The device of any one of claims 1 to 24, wherein the trigger port is an orifice formed in a wall of the body portion.

26. The device of any one of claims 1 to 25, wherein the output of the one or more sensors is received or obtained by a controller, which determines the status of the trigger port, and/or a change in the status of the trigger port based on the output.

27. The device of claim 26, wherein the controller controls operation of the respiratory apparatus such that the breathable gas is delivered to the patient at a suitable pressure.

28. The device of claim 27, wherein the controller controls operation of the respiratory apparatus by setting a motor speed of a flow generator.

29. The device of any one of claims 26 to 28, wherein the controller sends a control signal to the respiratory apparatus when there is a change in the status of trigger port.

30. The device of any one of claims 1 to 29, wherein the device includes a coupling mechanism allowing it to be removably connected to the patient interface.

31. The device of any one of claims 1 to 30, wherein the device receives the breathable gas from the respiratory apparatus via a conduit.

32. The device of claim 31, wherein the conduit is fluidly connected to the inlet of the device.

33. The device of any one of claims 1 to 32, wherein the device is a T-piece device.

34. An interface assembly for use in respiratory therapy, the interface assembly includes:
the device of any one of claims 1 to 33, and
a patient interface arranged to be in communication with an airway of a patient during respiratory therapy.

35. The device of any one of claims 1 to 33 or the interface assembly of claim 34, further comprising a conduit connected to an inlet of the device, arranged to be removably coupled to a conduit assembly of the respiratory apparatus.

36. A gas delivery system for delivering a breathable gas to a patient, comprising:
a respiratory apparatus including a flow generator, a controller arranged to control the flow generator, and one or more sensors configured to determine a source flow rate from the flow generator (Fs), and an interface gas flow rate (Fm);
a conduit assembly for delivering the breathable gas to the patient from the flow generator;
an interface assembly arranged to receive the breathable gas from the conduit assembly, and convey the breathable gas to the patient, wherein the interface assembly comprises a trigger port;
wherein the controller is arranged to: calculate a difference between the source flow rate and the interface gas flow rate, determine a status of the trigger port based on the difference, and operate the flow generator to provide the breathable gas to the patient at a pressure based on the determined status of the trigger port.

37. A method of controlling a gas delivery system, by:
determining a source gas flow;
determining a delivery gas flow;
calculating a difference between the source gas flow and the delivery gas flow, and
causing the gas delivery system to deliver a breathable gas to the patient at a pressure based on the calculated difference between the source and delivery gas flow.

38. A method of delivering a breathable gas to a patient via an interface assembly, said interface assembly including a trigger port, comprising:
determining a status of the trigger port, by determining a source gas flow generated by a gas delivery system and a delivery gas flow delivered to the interface assembly,
causing the gas delivery system to deliver a breathable gas to the patient at a pressure based on the determined status of the trigger port.

39. The method of claim 38, wherein the step of determining a source gas flow comprises:
receiving or obtaining measurements from one or more first sensors, and
determining a first flow rate based on the measurements, being the flow rate of source gas flow (Fs).

40. The method of claim 38 or 39, wherein the step of determining a delivery gas flow comprises:
receiving or obtaining measurements from one or more second sensors, and
determining a second flow rate based on the measurements, being the flow rate of the breathable gas supplied to a patient interface (Fm).

41. The method of claim 40, wherein the method further incudes: calculating a difference between the first and second flow rates (ΔF=Fs−Fm).

42. The method of claim 41, further including:
comparing the difference between the first and second flow rates with a first and/or a second threshold flow rate (Fa, Fb) and
determining a status of a trigger port based on the comparison.

43. The method of claim 42, wherein the status of the trigger port includes: an open state, or a closed state.

44. The method of claim 41, further including:
obtaining a previous status of a trigger port, wherein the previous status of the trigger port includes an open state or a closed state;
comparing the difference between the first and second flow rates with a first threshold flow rate Fa, if the previous status of the trigger port is in an open state, or comparing the difference between the first and second flow rates with a second threshold flow rate Fb, if the previous status of the trigger port is in a closed state,
determining a current status of the trigger port based on the comparison, and
determining if status of the trigger port has changed by comparing the previous status with the current status.

45. The method of any one of claims 42 to 44, wherein the trigger port is in the closed state if the difference is smaller than the first threshold flow rate (ΔF<Fa).

46. The method of any one of claims 42 to 45, wherein the trigger port is in the open state if the difference is greater than the second threshold flow rate (ΔF>Fb).

47. The method of any one of claims 42 to 46, wherein the second threshold flow rate is determined based on interface pressure, or calculated from K*sqrt(Pm), wherein Pm is interface pressure, and K is a coefficient determined experimentally or during a calibration stage.

48. The method of any one of claims 42 to 46, wherein the second threshold flow rate is within a range of 0.5 to 10 L/min.

49. The method of any one of claims 42 to 46, wherein the second threshold flow rate is set at a constant level.

50. The method of any one of claims 42 to 46, the second threshold flow rate is set at a constant level of 1 pm.

51. The method of any one of claims 42 to 50, wherein the first threshold flow rate is determined based on interface pressure, or calculated from Fa=J*sqrt(Pm), wherein Pm is interface pressure, and J is a coefficient determined experimentally or during a calibration stage.

52. The method of any one of claims 42 to 50, the first threshold flow rate is within a range of 0.5 to 10 L/min.

53. The method of any one of claims 42 to 50, wherein the first threshold flow rate is set at a constant level.

54. The method of any one of claims 42 to 50, wherein the first threshold flow rate is set at a constant level of 1 L/min.

55. The method of any one of claims 42 to 54, further including:
operating the gas delivery system to deliver a breathable gas to the patient at a first pressure, if the trigger port is determined to be in a closed state, and
operating the gas delivery system to deliver a breathable gas to the patient at a second pressure, if the trigger port is determined to be in an open state.

56. The method of any one of claims 42 to 55, further including:
operating the gas delivery system to continue to deliver the breathable gas to the patient at the first or a second pressure to the patient, until the status of the trigger port changes.

57. The method of any one of claim 55 or 56, wherein the first pressure is higher than the second pressure.

58. The method of any one of claims 55 to 57, wherein the first pressure corresponds to peak inspiratory pressure (PIP) and the second pressure corresponds to positive end expiratory pressure (PEEP).

59. The method of claim 58, wherein for an infant or neonatal patient, PEEP is equivalent to 1, 2, 3, 4, 5, 6, 7, or 8 cm H₂O, and PIP is equivalent to 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 cm H₂O.

60. The method of claim 56, wherein the step of operating the gas delivery system to deliver the breathable gas to the patient at the first or second pressure includes:
sending a control signal to a flow generator of the system.

61. The method of claim 60, wherein the flow generator is operated by controlling a motor speed of the flow generator.

62. The method of claim 60 or 61, wherein the flow generator is operated by controlling a speed of a blower fan.

63. The method of claim 61, wherein the motor speed is changed over a predetermined time interval until the delivered breathable gas reaches the first or second pressure flow.

64. The method of claim 63, wherein the predetermined time interval is between 100 to 400 ms, or between 100 to 300 ms, or between 100 to 200 ms, or approximately 150 ms.

65. The method of any one of claims 38 to 64, further including:
causing the trigger port to be in a closed state by substantially reducing or preventing air flow via the trigger port, and
causing the trigger port to be in an open state by allowing air flow via the trigger port.

66. The method of claim 65, wherein the step of causing the trigger port to be in a closed state comprises blocking the trigger port with an object, and the step of causing the trigger port to be in an open state comprises removing the object from the trigger port.

67. The method of claim 40, wherein the step of determining a second flow rate includes:
measuring a first pressure of the breathable gas at a first location;
measuring a second pressure of the breathable gas at a second location;
calculating a pressure differential between the first and the second pressure;
calculating the flow rate of the breathable gas supplied to the patient interface (Fm) based on the pressure differential.

68. The method of claim 67, wherein the first location is at or near an inlet of a device, where it receives the breathable gas from a conduit assembly in fluid communication with the flow generator.

69. The method of claim 67 or 68, wherein the second location is at or near an outlet of the device, where it conveys the breathable gas to the patient interface.

70. The method of any one of claims 67 to 69, wherein the second pressure is the interface pressure Pm.

71. A gas delivery system for delivering a breathable gas to a patient, comprising:
a respiratory apparatus including a flow generator, a controller arranged to control the flow generator, and at least one sensor proximate the flow generator, the at least one sensor configured to determine at least a source flow rate from the flow generator (Fs),
a conduit assembly for delivering the breathable gas to the patient from the flow generator;
an interface assembly arranged to receive the breathable gas from the conduit assembly, and convey the breathable gas to the patient, wherein the interface assembly comprises a trigger port;
wherein the controller is arranged to determine a status of the trigger port by comparing the determined source flow rate (Fs) to a threshold flow rate (Fth), and operate the flow generator to provide the breathable gas to the patient at a pressure based on the determined status of the trigger port.
